SEQUENCE LISTING (5 sequences)

SEQ ID NO: 1 - DNA, 8134 bp, synthetic construct
  source 1..8134: mol_type = other DNA
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattcttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcgcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttcccgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagcttatggctcgctcggtgaccctggtctttctggtgcttgtctcactgaccggcctgtatgctatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggtggaggtggatcaggtggatccggttctggaggtggagctagccagcaaaagaattacaccttccgctgcctgcagatgtcttcctttgcaaacagaagctggtcccgcacagacagcgtggtctggctgggggatctgcagactcaccgttggagtaatgactcagccaccatcagcttcacgaagccatggtcccagggcaagttgagtaaccagcagtgggagaagttgcagcatatgtttcaagtctatcgagtcagctttaccagggacatacaggaattagtcaaaatgatgtcacctaaagaagactatcccattgagatccagctgtctgcgggctgtgaaatgtaccctgggaatgcttcggaaagctttttacacgtagcatttcaaggaaaatatgtcgtgagattctggggaacgtcctggcagacagtcccaggggccccatcttggttagacttgcccatcaaagtgctcaacgctgatcaagggacaagtgcaaccgtgcagatgctcctgaatgacacctgccccctatttgtccgtggtctcctagaggcagggaagtcagacctagagaagcaagagaagccagtggcctggttgtccagtgtccccagctctgcagatggccataggcagctggtgtgtcatgtctctggcttctacccaaaacctgtgtgggtgatgtgcatgcggggtgaccaggagcaacagggtactcacagaggtgatttcctgccaaatgctgatgagacatggtatcttcaagcaaccctggatgtggaggctggagaggaagctggcctggcctgcagggtgaagcacagcagcctaggaggacaggatatcatcctctactgggatgccaggcaagcacccgtgggcggtggaggtggatcaggtggatccggttctggaggtggaggcgccgactctagaagcatcgtgatgacccagacccccaagttcctgctggtgagcgccggcgacagagtgaccatcacctgcaaggccagccagagcgtgagcaacgacgtgacctggtaccagcagaaggccggccagagccccaagctgctgatctacagcgccagcaacagatacagcggcgtgcccgacagattcaccggcagcggctacggcaccgccttcaccttcaccatcagcaccgtgcaggccgaggacctggccgtgtacttctgccagcaggactacagcagcttcggcggcggcaccaagctggagatcaagggtggcggtggctcgggcggtggtgggtcgggtggcggcggatctggtggcgggggttctcaggtgcagctgaaggagagcggccccggcctggtggcccccagccagagcctgagcatcacctgcaccgtgagcggcttcagcgtgaccaactacggcgtgcactgggtgagacagccccccggcaagggcctggagtggctgggcgtgatctgggccggcggcatcaccaactacaacagcgccttcatgagcagactgagcatcagcaaggacaacagcaagagccaggtgttcctgaagatgaacagcctgcagatcgacgacaccgccatgtactactgcgccagcagaggcggccactacggctacgccctggactactggggccagggcaccagcgtgaccgtgagcagcggttctggaggacatcatcatcaccaccattaagcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtcgcgcggtccttcgggcactcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctcggtgttcgaggccacacgcgtcaccttaatatgcgaagtggacctgggaccgcgccgccccgactgcatctgcgtgttaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 2 - DNA, 8179 bp, synthetic construct
  source 1..8179: mol_type = other DNA
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattcttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcgcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttcccgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagcttatggctcgctcggtgaccctggtctttctggtgcttgtctcactgaccggcctgtatgctatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggtggaggtggatcaggtggatccggttctggaggtggagctagccagcaaaagaattacaccttccgctgcctgcagatgtcttcctttgcaaacagaagctggtcccgcacagacagcgtggtctggctgggggatctgcagactcaccgttggagtaatgactcagccaccatcagcttcacgaagccatggtcccagggcaagttgagtaaccagcagtgggagaagttgcagcatatgtttcaagtctatcgagtcagctttaccagggacatacaggaattagtcaaaatgatgtcacctaaagaagactatcccattgagatccagctgtctgcgggctgtgaaatgtaccctgggaatgcttcggaaagctttttacacgtagcatttcaaggaaaatatgtcgtgagattctggggaacgtcctggcagacagtcccaggggccccatcttggttagacttgcccatcaaagtgctcaacgctgatcaagggacaagtgcaaccgtgcagatgctcctgaatgacacctgccccctatttgtccgtggtctcctagaggcagggaagtcagacctagagaagcaagagaagccagtggcctggttgtccagtgtccccagctctgcagatggccataggcagctggtgtgtcatgtctctggcttctacccaaaacctgtgtgggtgatgtgcatgcggggtgaccaggagcaacagggtactcacagaggtgatttcctgccaaatgctgatgagacatggtatcttcaagcaaccctggatgtggaggctggagaggaagctggcctggcctgcagggtgaagcacagcagcctaggaggacaggatatcatcctctactgggatgccaggcaagcacccgtgggcggtggaggtggatcaggtggatccggttctggaggtggaggcgccgactctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgacgctgcaccaactgtatccatcttcccaggtggcggtggctcgggcggtggtgggtcgggtggcggcggatctggtggcgggggttctgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatgaagtactggggtcaaggaacctcagtcaccgtctcctcaggttctggaggacatcatcatcaccaccattaagcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtcgcgcggtccttcgggcactcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctcggtgttcgaggccacacgcgtcaccttaatatgcgaagtggacctgggaccgcgccgccccgactgcatctgcgtgttaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 3 - DNA, 8179 bp, synthetic construct
  source 1..8179: mol_type = other DNA
ggcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcaagctagagtttaaacttgacagatgagacaataaccctgataaatgcttcaataatattgaaaaaggaaaagtatgagtattcaacatttccgtgtcgcccttattcccttttttgcggcattttgccttcctgtttttgctcacccagaaacgctggtgaaagtaaaagatgcagaagatcacttgggtgcgcgagtgggttacatcgaactggatctcaacagcggtaagatccttgagagttttcgccccgaagaacgtttcccaatgatgagcacttttaaagttctgctatgtggcgcggtattatcccgtattcttggttgaatactcaccagtcacagaaaagcatcttacggatggcatgacagtaagagaattatgcagtgctgccataaccatgagtgataacactgcggccaacttacttctgacaactatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgaaaactattaactggcgaactacttactctagcttcccggcaacaactaatagactggatggaggcggataaagttgcaggaccacttctgcgctcggcacttccggctggctggtttattgctgataaatcaggagccggtgagcgtgggtcacgcggtatcattgcagcactggggccggatggtaagccctcccgtatcgtagttatctacactacggggagtcaggcaactatggatgaacgaaatagacagatcgctgagataggtgcctcactgattaagcattggtaaggataaatttctggtaaggaggacacgtatggaagtgggcaagttggggaagccgtatccgttgctgaatctggcatatgtgggagtataagacgcgcagcgtcgcatcaggcatttttttctgcgccaatgcaaaaaggccatccgtcaggatggcctttcgcataactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttcccgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagcttatggctcgctcggtgaccctggtctttctggtgcttgtctcactgaccggcctgtatgctatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgaattcacccccactgagactgatacatacgcctgcagagttaagcatgccagtatggccgagcccaagaccgtctactgggatcgagacatgggtggaggtggatcaggtggatccggttctggaggtggagctagccagcaaaagaattacaccttccgctgcctgcagatgtcttcctttgcaaacagaagctggtcccgcacagacagcgtggtctggctgggggatctgcagactcaccgttggagtaatgactcagccaccatcagcttcacgaagccatggtcccagggcaagttgagtaaccagcagtgggagaagttgcagcatatgtttcaagtctatcgagtcagctttaccagggacatacaggaattagtcaaaatgatgtcacctaaagaagactatcccattgagatccagctgtctgcgggctgtgaaatgtaccctgggaatgcttcggaaagctttttacacgtagcatttcaaggaaaatatgtcgtgagattctggggaacgtcctggcagacagtcccaggggccccatcttggttagacttgcccatcaaagtgctcaacgctgatcaagggacaagtgcaaccgtgcagatgctcctgaatgacacctgccccctatttgtccgtggtctcctagaggcagggaagtcagacctagagaagcaagagaagccagtggcctggttgtccagtgtccccagctctgcagatggccataggcagctggtgtgtcatgtctctggcttctacccaaaacctgtgtgggtgatgtgcatgcggggtgaccaggagcaacagggtactcacagaggtgatttcctgccaaatgctgatgagacatggtatcttcaagcaaccctggatgtggaggctggagaggaagctggcctggcctgcagggtgaagcacagcagcctaggaggacaggatatcatcctctactgggatgccaggcaagcacccgtgggcggtggaggtggatcaggtggatccggttctggaggtggaggcgccgactctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgacgctgcaccaactgtatccatcttcccaggtggcggtggctcgggcggtggtgggtcgggtggcggcggatctggtggcgggggttctgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaggttctggaggacatcatcatcaccaccattaagcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtcgcgcggtccttcgggcactcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctcggtgttcgaggccacacgcgtcaccttaatatgcgaagtggacctgggaccgcgccgccccgactgcatctgcgtgttaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagatccatcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgacccccgctgcgcgggggcacgtcaggctcaccatctgggccgccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatgtgccatgatggcggcctgcagctgtgttcgaggccgcgcgtgtcaccttaatatgcgaagtggacctgggaccgcgccgccccgactgcatctgcgtgttcgagttcgccaatgacaagacgctgggcggggagatcccccttattaaccctaaacgggtagcatatgcttcccgggtagtagtatatactatccagactaaccctaattcaatagcatatgttacccaacgggaagcatatgctatcgaattagggttagtaaaagggtcctaaggaacagcgatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaaggagatctgtcgacatcgatgggcgcgggtgtacactccgcccatcccgcccctaactccgcccagttccgcccattctccgcctcatggctgactaattttttttatttatgcagaggccgaggccgcctcggcctctgagctattccagaagtagtgaggaggcttttttggaggcctaggcttttgcaaaaagctaattc

SEQ ID NO: 4 - DNA, 20 bp, synthetic construct
  source 1..20: mol_type = other DNA
tgcaaacagaagctggtccc

SEQ ID NO: 5 - DNA, 21 bp, synthetic construct
  source 1..21: mol_type = other DNA
acgtgtcagcacccggctggg

(19) United States
(12) Patent Application Publication
(10) Pub. No.: US 2026/0035479 A1
(43) Pub. Date: Feb. 5, 2026

(54) GD2 BI-SPECIFIC INVARIANT NATURAL KILLER T CELL ENGAGERS AND METHODS OF USE THEREOF

(71) Applicant: The Children's Hospital of Philadelphia, Philadelphia, PA (US)

(72) Inventors: Hamid BASSIRI, Philadelphia, PA (US); Kevin MCNERNEY, Philadelphia, PA (US)

(73) Assignee: The Children's Hospital of Philadelphia, Philadelphia, PA (US)

(21) Appl. No.: 19/099,890

(22) PCT Filed: Aug. 21, 2023
(86) PCT No.: PCT/US2023/072564

§ 371 Date: Jan. 30, 2025

Related U.S. Application Data
(60) Provisional application No. 63/399,484, filed on Aug. 19, 2022.

(51) Int. Cl. C07K 16/30; C07K 14/705; C07K 14/74

(52) U.S. Cl. CPC C07K 16/3084; C07K 14/70503; C07K 14/70539; C07K 2317/622; C07K 2319/32

(57) ABSTRACT

The present disclosure is directed to compositions comprising fusion proteins of CD1d and anti-GD2 antibody that may be used as GD2-specific Bi-specific invariant natural killer T cell engagers (BiNTEs). Further provided herein are methods of using the fusion proteins or BiNTEs for the treatment of cancer.

### PRIORITY CLAIM

This application claims benefit of priority to U.S. Provisional Application Ser. No. 63/399,484, filed Aug. 19, 2022, the entire contents of which is hereby incorporated by reference.

### STATEMENT REGARDING FEDERALLY FUNDED RESEARCH

This invention was made with government support under Grant No. T L1-T R001880, awarded by the National Institutes of Health. The government has certain rights in the invention.

### SEQUENCE LISTING

This application contains a Sequence Listing XML, which has been submitted electronically and is hereby incorporated by reference in its entirety. Said XML Sequence Listing, created on Aug. 4, 2023, is named CHOPP0061WO.xml and is 29,732 bytes in size.

### BACKGROUND

### 1. Field of the Disclosure

The present disclosure relates generally to the fields of immunology and cancer immunotherapy. More particular, the disclosure relates to compositions and methods for the treatment of solid tumors including neuroblastoma using molecules that activate invariant natural killer T cells (iNKTs).

### 2. Background

Neuroblastoma (NB) is the most common extracranial solid tumor in children and accounts for approximately 15% of childhood cancer deaths (Coughlan et al., 2017). Current treatments for high-risk NB (HRNB) include aggressive chemotherapy, autologous transplant, radiation, and most recently, immunotherapy. The immunotherapy regimen for high-risk neuroblastoma patients includes the use of a chimeric antibody against a neuroblastoma tumor specific antigen, the disialoganglioside GD2. This antibody is called dinutuximab and has shown improved EFS and disease responsiveness in high risk and relapsed and refractory neuroblastoma patients (Mody et al., 2017; Yu et al., 2010). Current treatment regimens for HRNB attain 5-year overall-survival (OS) rates of approximately 50%, and are associated with numerous late effects including hearing loss, cognitive deficits, endocrinopathies, and ovarian failure (Coughlan et al., 2017; Friedman & Henderson, 2018; Laverdiere et al., 2005; Portwine et al., 2016). As such, there is a critical need for more tolerable and effective treatments in this group.

### SUMMARY

In certain embodiments, the present disclosure provides GD2-specific Bi-specific invariant natural killer T cell engagers (BiNTEs, also referred to herein as 2^{nd} generation Cabs). In a first embodiment, there is provided a CD1d polypeptide conjugated to an anti-GD2 single chain variable fragment (scFv).

In some aspects, the composition further comprises a β2-microglobulin (β2m) polypeptide or fragment thereof. In some aspects, the CD1d polypeptide is a soluble CD1d polypeptide or fragment thereof.

In certain aspects, the anti-GD2 scFV has at least 90% (e.g., 91%, 92%, 93%, 94%, 95%, 96%, 97%, 98%, or 99%) sequence identity to the 14G2a GD2 scFv, E101K GD2 scFv, or 3F8 GD2 scFv. In certain aspects, the anti-GD2 scFV comprises the 14G2a GD2 scFv, E101K GD2 scFv, or 3F8 GD2 scFv.

In some aspects, the composition further comprises a peptide linker between the CD1d polypeptide and anti-GD2 scFv. In some aspects, the composition further comprises a peptide linker between the β2m polypeptide and the CD1d polypeptide. In certain aspects, the peptide linker is a Glycine-Serine (GS) linker. For example, the peptide linker is G₁₀S₃. Glycine serine linkers can be adjusted for flexibility by varying glycine and serine compositions and length of peptide linker. In particular aspects, the composition does not comprise a streptavidin-biotin linker, the CD1d polypeptide is not biotinylated.

In some aspects, the composition further comprises a glycolipid antigen. In some aspects, the glycolipid antigen is a non-phenylated glycolipid antigen. In certain aspects, the non-phenylated glycolipid antigen is α-GalactosylCeramide (aGC). In particular aspects, the glycolipid antigen is a phenylated glycolipid antigen. In some aspects, the phenylated glycolipid antigen is phenyl α-GalactosylCeramide (C34).

Further provided herein is a polynucleotide encoding the composition of the present embodiments (e.g., a CD1d polypeptide conjugated to an anti-GD2 single chain variable fragment (scFv)) and aspects thereof. In some aspects, the polynucleotide encodes from N-terminal to C-terminal the β2m polypeptide, the CD1d polypeptide, and the anti-GD2 scFv. In particular aspects, the polynucleotide has at least 90% (e.g., 91%, 92%, 93%, 94%, 95%, 96%, 97%, 98%, or 99%) sequence identity to SEQ ID NO:1, 2, or 3. In certain aspects, the polynucleotide comprises SEQ ID NO:1, 2, or 3.

In a further embodiment, there is provided an expression vector comprising the polynucleotide of the present embodiments and aspects thereof.

Another embodiment provides a host cell comprising an expression vector of the present embodiments. In some aspects, the host cell is an antigen presenting cell (APC), such as a dendritic cell.

A further embodiment provides a method of stimulating immune cells comprising said immune cells with the composition of the present embodiments or aspects thereof or a host cell of the present embodiments or aspects thereof. In some aspects, the immune cells are invariant natural killer T cells (iNKTs). In certain aspects, the iNKTs are stimulated for less than 24 hours. In some aspects, the iNKTs are stimulated for 1-10 days. In some aspects, the iNKTs are stimulated more than once. In certain aspects, the iNKTs are stimulated two, three, or four times. In some aspects, the iNKTs are further stimulated with one or more cytokines. In certain aspects, the one or more cytokines are IL-12 and/or IL-18.

Further provided herein is a population of GD2-targeted iNKTs stimulated by the composition of the present embodiments and aspects thereof.

Another embodiment provides a pharmaceutical composition comprising the composition of any of the present embodiments and aspects thereof and a pharmaceutically acceptable carrier.

A further embodiment provides a composition according to the any of the present embodiments and aspects thereof for use in the treatment of cancer in a subject.

In yet another embodiment, there is provided a method for treating cancer in a subject comprising administering an effective amount of the composition (e.g., a GD2-specific BiNTE) of any of the present embodiments and aspects thereof to the subject.

In some aspects, the cancer is neuroblastoma, osteosarcoma, Ewing's sarcoma, and melanoma. In certain aspects, the method further comprises administering to said subject at least a second anti-cancer therapy. In some aspects, the second anti-cancer therapy is chemotherapy, radiotherapy, gene therapy, surgery, hormonal therapy, anti-angiogenic therapy or immunotherapy. In certain aspects, the second anti-cancer therapy is a monoclonal antibody, CAR T-cell therapy, or a second bispecific T-cell engager. In some aspects, the subject is human.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1B: Depiction of GD2-CAb and GD2-BiNTE proteins. (FIG. 1A) Left side of figure depicts a CAb with anti-GD2 antibody (black), CD1d (purple), glycolipid antigen (red circle), and streptavidin/biotin linker (maroon crescent and green circle). The GD2-CAb protein engages the iNKT cell TCR (dark green) via the CD1d/glycolipid antigen complex, while simultaneously engaging GD2 (light grey) on the surface of tumor cells such as neuroblastoma (NB) via the anti-GD2 antibody. Right side of figure depicts a GD2-BiNTE with a GD2-specific single chain variable fragment (scFv; black) and a CD1d molecule (purple) harboring a glycolipid antigen (red circle). The GD2-BiNTE engages the iNKT cell TCR (dark green) via the CD1d/glycolipid antigen complex, while simultaneously engaging GD2 (light grey) on the surface of tumor cells such as neuroblastoma (NB) via the anti-GD2 scFv. (FIG. 1B) Proposed action of CAb and BiNTE (also known as 2^{nd} generation CAb) proteins. Left panel depicts a CAb with 14G2a anti-GD2 antibody (dark blue), CD1d (tan), aGC (salmon), and streptavidin/biotin linker (white). iNKTs release cytokines that potentiate CD8+ T cells and NK cells, and mediate direct NB cytotoxicity. Right panel depicts a BiNTE (2^{nd}-generation CAb) without the streptavidin-biotin linker, scFv against GD2 (navy), loaded with the glycolipid antigen C34 (red).

FIG. 2: 1^{st} Generation GD2-CAb vs GD2+ NB cell line. Sorted murine iNKTs (effectors) were co-incubated for 18 hours in triplicate with 5Chromium-treated GD2+ NB cell line 273 (targets) in the presence of a 1^{st} generation GAg-loaded anti-GD2 CAb (pink), unloaded anti-GD2-CAb (orange), with alpha galactosyl ceramide (a-GalCer) alone (blue), and without stimuli (black) and percent specific lysis (y-axis) was calculated. Effector to target (E:T) ratios shown on x-axis.

FIGS. 3A-3C: Map of original pEAK-4D5 plasmid, synthesized insert, and modified fusion genes. (FIG. 3A) The pEAK8-β2M-CD1d-anti-HER2 (4D5) plasmid map containing ampicillin resistance, β2m, soluble CD1d (sCD1d), 4D5 scFv, and 6×His sequence. (FIG. 3B) Sequence of insert of CD1d shown with XbaI restriction site located in between the CD1d and E101K scFv sequences. (FIG. 3C) Schematic for modification of the original 4D5-containing plasmid to contain GD2, 3F8, and E101K scFvs.

FIGS. 4A-4B: Size of 3F8 and GD2 scFv PCR products and pEAK8 plasmids. (FIG. 4A) GeneRuler (1 Kb DNA Ladder) PCR products for 3F8 and GD2 inserts. (FIG. 4B) 1 KbGeneRulerPlus, pEAK8-4D5, pEAK8-E101K, pEAK8-3F8, and pEAK8-GD2 plasmids.

FIG. 5: Transfection efficiency of HEK293T cells with PEI-transfection. GFP-expression measured under fluorescence microscope with green fluorescent protein (GFP) capture in the left panel, and GFP overlay with light microscopy in the right panel.

FIG. 6: Purification of β2M-CD1d-antiGD2 fusion proteins. Lane 1: Dual Color DNA ladder with specified markers. E101K=β2m-CD1d-E101K-6×His; GD2=β2m-CD1d-GD2-6×His; RT=Run through; W=Wash; E=Elution.

FIG. 7: iNKT Response to Glycolipid Antigen Stimulation. Two C57BL/6 mice per group were injected with phosphate-buffered saline (PBS), alpha-galactosyl ceramide (a-GalCer), or C34 at hour 0. At hour 4, mice were euthanized, their livers were excised and hepatic lymphocytes isolated and stained for NK1.1+ TCR-β+ mononuclear single cells, and intracellular IFN-γ was assayed by flow cytometry. The IFN-γ positive gate was drawn based on unstained controls. Representative data shown are from one mouse.

FIGS. 8A-8C: Acute and Chronic cytokine expression following GAg-stimulation of iNKTs. Two C57BL/6 mice per group were injected with 4 μg of C34, alpha galactosyl-ceramide (aGC), or Phosphate-Buffered Saline (PBS), in acute or chronic stimulation groups. Hepatic lymphocytes were then isolated and stained for NK1.1, TCR-β, and interferon-gamma (IFN-γ) with intra- and extracellular staining methods. (FIG. 8A) IFN-γ mean fluorescence intensity (MFI) of iNKTs. (FIG. 8B) CD107a MFI of iNKTs. (FIG. 8C) Interleukin-4 MFI of iNKTs.

FIGS. 9A-9B: iNKT-mediated cytotoxicity with glycolipid antigen (GAg) stimulation. (FIG. 9A) C57BL/6 hepatic lymphocytes (effectors) were co-cultured for 18 hours with ⁵¹Cr-loaded EL4 lymphoma cells (targets) in triplicates at E:T ratios of 2.5:1, 5:1,10:1, 20:1, and 40:1; these cocultures were stimulated with either alpha galactosyl-ceramide (a-GalCer) or C34 at indicated concentrations or left unstimulated (Nothing added). (FIG. 9B) Similar experiments were performed with flow cytometrically sorted murine hepatic iNKTs as effector populations.

FIG. 10: iNKTs and interactions with immune effectors. iNKTs interact with members of the immune system, including tumor associated macrophage (TAM), myeloid derived suppressor cells (MDSC), neutrophils, NKs, T cells, and B cells. These interactions occur via their T cell receptor (TCR) binding to CD1d complexes bearing glycolipid antigens (GAg), through CD40/CD40L binding, and/or through cytokine release. Dendritic cells are matured by iNKTs via CD40L/CD40 binding and the release of IFN-γ, which then stimulates IL-12 release and the reciprocal stimulation of iNKTs. IFN-γ and IL-12 also stimulate nearby NKs, T cells, and B cells, helping to generate a pro-inflammatory immune environment. TAMs are reprogrammed from M2 macrophage to more pro-inflammatory M1 macrophage by GM-CSF release. MDSC modulation by iNKTs reduces the inhibition of T cell function.

FIG. 11: Interactions of iNKTs, NKs, and the TME. iNKTs and NKs are inhibited through the release of TGF-β, IL-4, IL-6, IL-10, IL-13, adenosine, and PGE-2. IL-6 also stimulates the growth of neuroblasts and increases osteoclastic activity associated with metastatic potential. TGF-β reduces expression of the activating NK receptor, NKG2D, limiting NK activation. MYCN expressed in neuroblasts downregulates MHC-I expression and ligands for NK-activating receptors including DNAM-1 and NKG2D ligands, thereby promoting immune evasion. Galectin-3, HLA-G, and PD-L1 expression also contribute to immune evasion or downregulation in the TME.

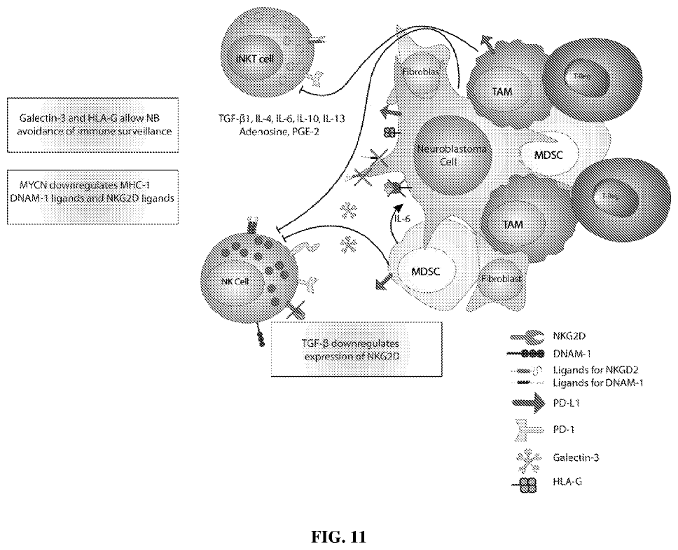

FIGS. 12A-12D: iNKT-based NB Treatments. NB therapies using iNKTs may employ administration of glycolipid antigens (GAg). GAg loaded onto soluble CD1d, or GAg-pulsed dendritic cells (FIG. 12A), adoptive transfer of previously stimulated iNKTs (FIG. 12B), or CAR-modified iNKTs (FIG. 12C), and combined with PD-1/PD-L1 inhibition (FIG. 12D).

FIG. 13: Structures of aGC and C34 glycolipids.

### DESCRIPTION OF ILLUSTRATIVE EMBODIMENTS

Invariant natural killer T-cells (INKTs) are immune cells whose frequency is associated with improved survival in NB patients as well as other pediatric and adult malignancies (Guillerey et al., 2016; Hishiki et al., 2018; Krasnova et al., 2017; Wolf et al., 2018). They are innate-like lymphocytes that recognize and are activated by an MHC-I-like protein called CD1d, which presents glycolipid antigens (GAgs). Upon activation, iNKTs are capable of releasing copious cytokines which recruit immune effectors and reprogram immunosuppressive cells in the neuroblastoma tumor microenvironment (Song et al., 2009).

iNKT therapies include iNKT stimulation with free or CD1d-associated glycolipid antigens, iNKT-adoptive cell transfer, and chimeric antigen receptor (CAR)-iNKTs. However, iNKT therapies have been limited by the development of iNKT anergy, which is defined as a diminished function (e.g., cytokine release and proliferation) on repeated stimulation. In fact, mice exposed to glycolipid antigens can have evidence of iNKT anergy for up to 6 months following stimulation with the most commonly studied glycolipid antigen, α-GalactosylCeramide (aGC) (Parekh, 2005).

The present studies were directed to address this major limitation with iNKT activation for antitumor toxicity via 2 approaches: (1) utilization of a phenylated glycolipid antigen with high affinity binding to the iNKT T cell receptor (TCR), and (2) development of a fusion protein that links CD1d and several single-chain variable fragments (scFvs) against GD2 for iNKT stimulation and NB tumor-targeting via GD2 recognition.

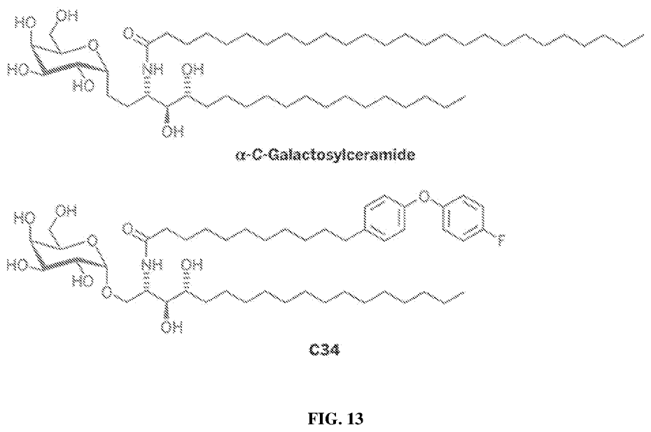

Specifically, the effects of a non-phenylated and phenylated GAg (aGC and C34 (phenyl α-GalCer), respectively, structures are depicted in FIG. 13) were explored on the cytokine production of iNKT cells after acute and chronic in vivo stimulation, and the influence of these GAgs on iNKT-mediated cytotoxicity in killing assays. Mice were injected with aGC and C34, in “acute” and “chronic” stimulation scenarios and hepatic iNKTs were stained and analyzed for cytokine expression by flow cytometry. iNKT-mediated cytotoxicity of EL4 lymphoma cell lines was measured with ⁵¹Cr-release killing assays. The C34 stimulation showed reduced iNKT anergy, increased iNKT IFN-γ release, and robust iNKT-mediated tumor-specific cytotoxicity. Thus, in some embodiments, methods are provided for the use of C34 in promoting an iNKT-mediated anti-tumor response.

Invariant natural killer T (iNKT) cells are T lymphocytes that bear certain NK cell biomarkers. iNKT cells express a restricted (hence “invariant”) set of T cell receptors, iNKT cells recognize and are activated by the MHC-I-like molecule CD1d, which specializes in the presentation of glycolipid antigens (GAgs). CD1d/GAg complexes can potently activate iNKTs and enable antitumor functions. iNKTs can be further targeted to specific tumors by employing single chain variable fragment (scFv) proteins, which are small proteins comprised of antibody heavy and light chain fragments connected by a short linker peptide. scFvs specific for tumor associated antigens (TAA) have been used to construct a variety of chimeric antigen receptor (CAR)-expressing T cells.

Accordingly, in further embodiments, the present disclosure provides fusion proteins composed of CD1d molecules attached to a variety of scFv with specificity for the disialoganglioside GD2 (a TAA for the pediatric cancer neuroblastoma). These fusion products can be used to activate and target iNKT cells to GD2-expressing neuroblastoma cells.

Specifically, a construct comprising a beta-2-microglobulin (b2m) molecule fused to a single soluble CD1d (sCD1d) molecule was attached to one of 3 different anti-GD2 scFV genes (“GD2” which encodes for the scFv from the 14G2a clone of anti-GD2 mAb, “E101K” which encodes for a single amino acid substitution variant of the 14G2a allowing for increased affinity to GD2, and “3F8” which encodes for an scFv of another anti-GD2 mAb called clone 3F8). Three independent b2m-sCD1d-anti-GD2 fusion constructs whose genetic sequences were confirmed by Sanger sequencing: pEAK8-b2m-sCD1d-E101K-6×His (SEQ ID NO:2), pEAK8-b2m-sCD1d-3F8-6×His (SEQ ID NO:1), and pEAK8-b2m-sCD1d-GD2-6×His (SEQ ID NO:3).

These and other aspects of the disclosure are described in detail below.

### I. INVARIANT NATURAL KILLER T CELLS

iNKTs have been studied extensively pre-clinically in the solid tumor environment due to the improved prognosis that they have been associated with in various solid tumors, including NB (Guillerey et al., 2016; Wolf et al., 2018). Activated iNKTs inhibit the immunosuppressive effects of tumor associated macrophages (TAMs) and myeloid derived suppressor cells (MDSCs), mediate direct cellular cytotoxicity, and release large amounts of cytokines that direct NK and cytotoxic T-cell mediated lysis (Kmieciak et al., 2011). Interestingly, iNKTs have been shown to enhance NK-mediated antibody dependent cellular cytotoxicity (ADCC) of neuroblasts exposed to dinutuximab and have also been shown to rescue exhausted T-cell cytotoxicity in PD-1 inhibitor resistant cancer models (Misc et al., 2016; Bca et al., 2018).

Interestingly, the presence of iNKTs is associated with improved prognosis of patients with NB as well as other malignancies (Guillerey et al., 2016; Hishiki et al., 2018; Krasnova et al., 2017; Wolf et al., 2018). iNKTs inhibit tumor-associated macrophages (TAMs) and myeloid derived suppressor cells (MDSCs) and robustly secrete cytokines to recruit additional immune effectors. These features allow iNKTs to decrease tumor immunosuppression and overcome immune evasion in the HRNB TME (Kmieciak et al., 2011). Thus, iNKT-based immunotherapies may prove useful in both stand-alone and combination treatments for NB and other pediatric solid tumors.

One mechanism by which iNKTs are activated is via ligation of their invariant TCR with an MHC-I-like protein named CD1d, which presents glycolipid antigens. When iNKTs are activated, they release large amounts of cytokines that mature, recruit, and activate other immune effector cells. Similar to conventional CD4+ T cells, iNKTs produce Th1-, Th2-, and Th17-like cytokines. Th-1 cytokines such as IFN-γ are associated with pro-inflammatory responses and enhanced antitumor cytotoxicity, whereas Th-2 cytokines (e.g., IL-4 and IL-10) promote immune tolerance and limit autoimmunity (Hung et al., 2017). The avidity and stability of the CD1d-GAg complex influences the ratio of Th1:Th2 cytokines released (Hung et al., 2017; Fujio et al., 2006; Huang et al., 2014). CD1d-GAg complexes with high affinity for the iNKT TCR induce less iNKT anergy when compared with lower affinity CD1d-GAg complexes (Hung et al., 2017). Anergy of iNKTs limits iNKT-mediated tumor cytotoxicity and Th1-like cytokine release (Hung et al., 2017). Additionally, stimulation of iNKTs with GAgs of low TCR affinity has been shown to lead to accumulation of MDSCs in tumors and has resulted in worsened pulmonary metastases in a xenograft mouse model of melanoma via a presumed mechanism of increased Th-2 cytokine release from anergic iNKTs with ongoing stimulation (Parekh, 2005; Huang et al., 2014). These observations illustrate the importance of the GAg in mediating iNKT stimulation (Parekh, 2005; Huang et al., 2014).

NKTs are innate-like lymphocytes that make up about 1% of total lymphocytes in the human liver, and also have residence in the spleen and bone marrow. They bridge the innate and adaptive immune systems, helping to coordinate robust responses to malignant or infected cells, and have demonstrated importance in tumor immunosurveillance (Bassiri et al., 2013; Crowe et al., 2002; Cui et al., 1997). NKTs share features with both NKs and T cells. Type I, or invariant NKTs (iNKTs), express a conserved T cell receptor (TCR) made up of an α chain composed of Vα24 and Jα18 segments paired with a β chain composed of the VB11 segment. Conversely, type II NKTs express polyclonal TCRs, similar to conventional CD4+ and CD8+ T cells. The invariant TCRs on iNKTs allow for the recognition of glycolipid antigens (GAgs) presented by a non-polymorphic and conserved MHC class 1-like protein called CD1d (Table 1).

TABLE 1
Features of human type I and type II NKTs

| Feature | Type I NKTs (iNKTs) | Type II NKTs |
|---|---|---|
| Human TCR components | Vα24, Jα18, Vβ11 | Polyclonal, Vα3.2, Vα8, Vα1, Vβ8, Vβ3 |
| TCR recognizes | GAg-loaded CD1d | Sulfatides/lyophospholipid-loaded CD1d |
| Activated by aGC/CD1d complex | Yes | No |
| Phenotype | CD161+, CD4+/−, CD8+/−, or DN | CD161+, CD4+/−, CD8+/−, or DN |
| Cytokine production | IFN-γ, IL-4, IL-3 | IL-4, IL-13, IL-10, IFN-γ |

Abbreviations: Natural killer T cell (NKT), invariant natural killer T cell (iNKT), T cell Receptor (TCR), Glycolipid antigen (GAg), double-negative (DN), α-GalactosylCeramide (aGC), interferon-gamma (IFN-γ), interleukin (IL) (Waldowska et al., 2017).

The frequency of iNKTs within a tumor, and in peripheral circulation, has been associated with improved survival and reduced progression in various malignancies including prostate cancer, medulloblastoma, melanoma, multiple myeloma, colon cancer, lung cancer, breast cancer, head and neck squamous cell carcinomas (HNSCC), and NB (Wolf et al., 2018; Molling et al., 2007; Exley et al., 2011). Additionally, lack of function in iNKTs is associated with advanced cancers and worse prognosis in patients with multiple myeloma, multiple myeloma, myelodysplastic syndrome, and prostate cancer (Dhodapkar et al., 2003; Fujii et al., 2003; Tahir et al., 2001; Berzins et al., 2011). In xenograft models for NB, mice lacking iNKTs had more metastases and shortened survival in comparison to their iNKT-replete counterparts (Courtney et al., 2017). In another study, absence of iNKTs in mice lacking one allele of the tumor suppressor p53 were predisposed to earlier development of a variety of cancers and to decreased survival (Swann et al., 2009). Finally, for NB patients at the time of diagnosis, a high frequency of iNKTs in NB tumors was found to be associated with improved survival and lower stage NB (Hishiki et al., 2018). Given the apparent importance of iNKTs in tumor immunology, elucidating the various modes of iNKT activation and activity is of great interest.

### A. iNKT Activation and Activity

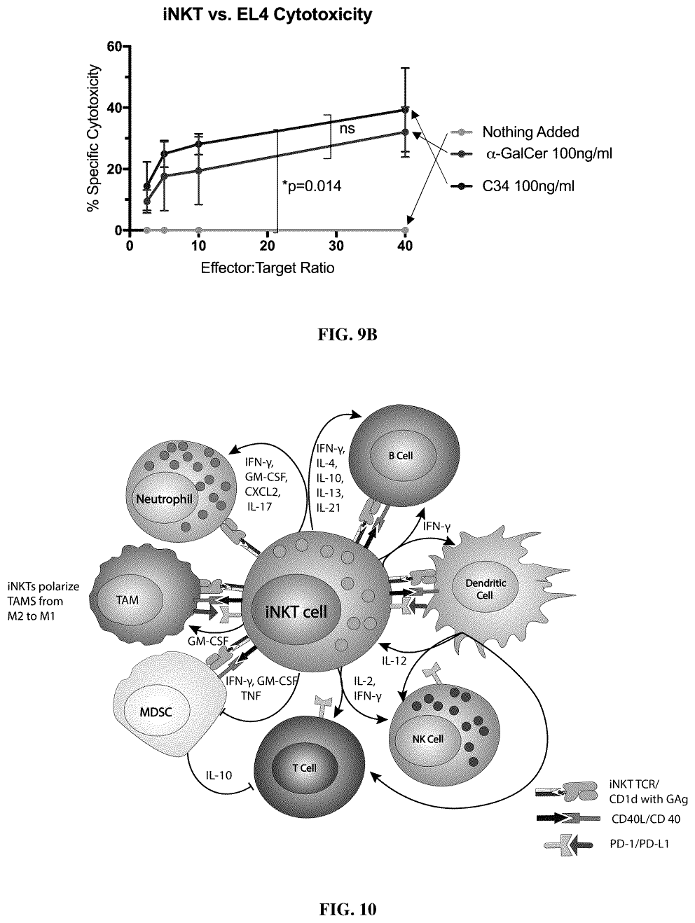

iNKTs can be activated in CD1d-dependent and -independent manners (FIG. 10). As mentioned above, the iNKT TCR binds to GAg presented by CD1d proteins, which are expressed by most cells of hematopoietic origin including TAMs and MDSCs. Cells that are transformed or infected present immunogenic GAgs, or have changes in their actin cytoskeleton that create CD1d nanoclusters of high avidity, leading to iNKT activation (Bedard et al., 2017; Brennan et al., 2011; Torreno-Pina et al., 2016). Independent of their CD1d-driven activation. iNKTs can also be activated through exposure to the cytokines IL-12 and IL-18 (Reilly et al., 2010; Tyznik et al., 2008; Wesley et al., 2008). Additionally, co-stimulatory signals from CD28, 4-1BB (CD137), NKG2D, CD40L, and ICOS (CD278) mediate robust iNKT activation (Vinay et al., 2004; Wang et al., 2009; Wang et al., 2013; Kaneda et al., 2005; Kuylenstierna et al., 2011). Dendritic cells (DCs) use cytokine release (IL-12) and cell-cell signaling with iNKTs through GAg-CD1d/TCR, and CD40L/CD40 binding are of particular importance for iNKT activation. Notably, the production of cytokines IFN-γ and IL-12 by the activated iNKTs and DCs results in enhanced NK- and T-cell mediated antitumor responses (Wolf et al., 2018; King et al., 2018; Kitamura et al., 1999; Nair & Dhodapkar, 2017).

When iNKTs are activated, they release large amounts of cytokines that mature, recruit, and activate other immune effector cells. Similar to conventional CD4+ T cells, iNKTs produce Th1-, Th2-, and Th17-like cytokines. Th-1 cytokines arc associated with pro-inflammatory responses and enhanced antitumor cytotoxicity, whereas Th-2 cytokines promote immune tolerance and are useful in limiting autoimmunity (Hung et al., 2017). The avidity and stability of the CD1d-GAg complex influence the ratio of Th1:Th2 cytokines released (Hung et al., 2017; Fujio et al., 2006; Huang et al., 2014). Furthermore, CD1d-GAg complexes with high affinity for the iNKT TCR have been shown to limit accumulation of immunosuppressive MDSCs in tumors when compared with lower affinity CD1d-GAg complexes. Higher affinity CD1d-GAg complexes also induce less iNKT anergy, which is defined by a lack of activation on repeated stimulation (Hung et al., 2017; Fujio et al., 2006). These observations illustrate the importance of the type of GAg/CD1d complex mediating iNKT stimulation (Parekh, 2005; Huang et al., 2014).

In addition to iNKT cytokine production, iNKTs can also recognize, kill, and reprogram CD1d+ cells including immunosuppressive TAMs and MDSCs. They mediate cytotoxic responses through release of perforin/granzyme B, or through upregulation of Fas ligand (FasL; CD178) and TNF-related apoptosis inducing ligand (TRAIL; CD253) (Bassiri et al., 2013; Nair & Dhodapkar, 2017). iNKTs also produce granulocyte-macrophage colony stimulating factor (GM-CSF), which reprograms TAMs to display a pro-inflammatory (M1) phenotype (Song et al., 2009; Courtney et al., 2017; Metelitsa, 2011). The ability to kill or reprogram TAMs is crucial, as expression of TAM-specific genes in the NB TME is associated with poor 5-year FFS (Song et al., 2009). TAMs are not only immunosuppressive, but also promote neuroblast growth and increased osteoclastic activity associated with bone metastases through release of IL-6 (Song et al., 2009; Sohara et al., 2005). Notably, iNKTs may be the only known effector cells that recognize and negatively regulate TAMs (Liu et al., 2012). iNKTs are also capable of culling IL-10-producing immunosuppressive MDSCs, which can limit suppression of cytotoxic T cells, resulting in improved tumor control (Mussai et al., 2012).

### B. Anergy and Tumor Microenvironment Immunosuppression of iNKT Cells

Despite their many favorable features, certain barriers face the use of iNKTs as cancer immunotherapeutics. One such limitation is that of anergy, a state in which iNKTs fail to produce cytokines or proliferate after stimulation. Indeed, murine iNKTs have been shown to become anergic for periods of up to six months after a single exposure to aGC, the highly potent canonical GAg used to stimulate iNKTs in numerous studies (Parekh, 2005). iNKT anergy not only limited anti-tumor activity, but further stimulation of anergic iNKTs actually worsened tumor metastasis in a mouse model of melanoma (Parekh, 2005). In human iNKTs, anergy with aGC stimulation has also been shown to be a T-regulatory cell and DC-dependent process using co-culture experiments with peripheral blood mononuclear cell-derived iNKTs (Ihara et al., 2019). iNKTs isolated from patients with advanced multiple myeloma were also shown to have significantly diminished IFN-Y production on aGC stimulation (Dhodapkar et al., 2003).

Similar to conventional T cells, an additional barrier can be imposed by proteins expressed on neuroblasts, TAMs, MDSCs, and regulatory T cells (TREG). Proteins such as programmed cell death ligand-1 (PD-L1) binds to PD1 on iNKTs and other immune effectors to inhibit their cytotoxic function (Beldi-Ferchiou & Caillat-Zucman, 2017; Bellucci et al., 2015; Chang et al., 2008; Hsu et al., 2018; Parekh et al., 2009; Durgan et al., 2011). Aside from expression of checkpoint ligands such as PD-L1, neuroblasts and co-opted immune cells also release TGF-β1, IL-4, IL-6, IL-10, IL-13, adenosine, and prostaglandin E-2 to suppress infiltrating immune cells (Song et al., 2009; Sohara et al., 2005). This immunosuppressive milieu can bias iNKTs towards Th2 cytokine release, thereby skewing the TME in an immunosuppressive direction.

These barriers have been targeted using strategies to reduce anergy and block checkpoint pathway signaling. Anergy has been shown to be inhibited by pulsing DCs with GAg, or loading soluble CD1d with synthetic GAgs modified for greater CD1d/TCR affinity. Indeed, the C-glycoside analog of aGC and phenylated GAgs activate and skew iNKTs towards Th1-cytokine secretion, thereby counteracting the immunosuppressive cytokines of the TME (Huang et al., 2014). Phenylated GAgs also limit the anergy experienced by iNKTs on repeated stimulation and result in less accumulation of MDSCs in the TME than aGC, suggesting the advantage of their use for iNKT stimulation (Huang et al., 2014). Finally, the immunosuppressive effects of checkpoint receptor expression have been targeted by antibody-mediated blockade of PD-1/PD-L1 interactions; this restores the IFN-γ release and augments anti-tumor activity of iNKTs (Wang et al., 2013; Chang et al., 2008; Parekh et al., 2009; Durgan et al., 2011; Guo et al., 2016).

iNKTs are associated with favorable prognosis in various human malignancies, likely due to their ability to secrete pro-inflammatory cytokines and culling and/or reprogramming of immunosuppressive and tumor-growth promoting cells in the TME. As such, therapies that minimize iNKT anergy and promote the release of Th1 over Th2 cytokines as provided herein can have greater antitumor efficacy.

### C. iNKT-Based Treatments of NB

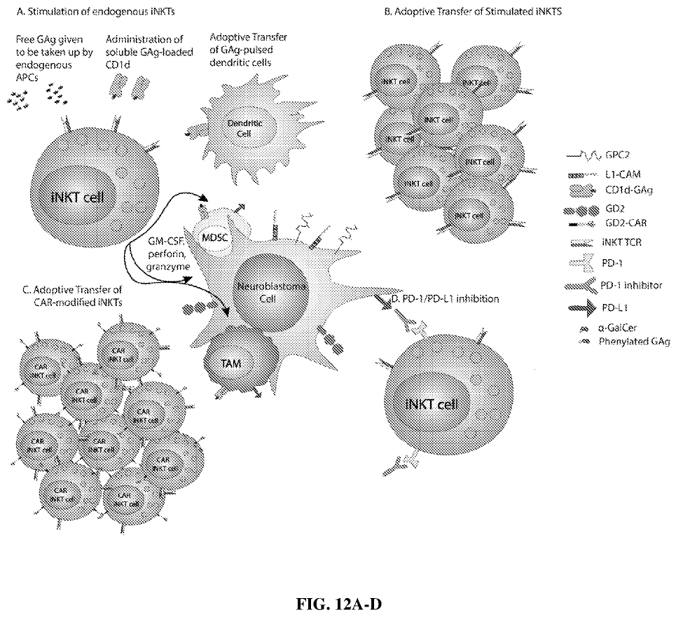

There has been an accumulation of preclinical and early phase clinical data indicating that iNKT-based immunotherapies may have promise in the treatment of NB. Some approaches employing iNKTs for immunotherapy of NB include GAg stimulation of iNKTs, adoptive transfer of iNKTs, and CAR-iNKTs (FIG. 12).

### D. GAg Stimulation of iNKT Cells

iNKTs can be stimulated for antitumor therapy by providing GAg alone, or GAg-loaded antigen-presenting cells (APCs) that could promote activation of endogenous iNKTs. These strategies have been used in patients with various solid tumors, with modest but encouraging results. For example, in a Phase I trial evaluating the effects of iNKT stimulation with aGC in patients with advanced solid tumors, serum GM-CSF and TNF-α were increased in 5 of 24 patients treated, albeit without disease response. Significantly, the patients that had a cytokine bump from the aGC had higher levels of pre-treatment iNKTs, and yet no dose-limiting toxicities were noted (Table 2) (Giaccone et al., 2002). Another strategy for GAg-stimulation of iNKTs involves pulsing DCs with aGC, and administering these pulsed DCs intravenously. In various trials of patients with non-NB solid tumors, this strategy led to decreases in tumor markers, iNKT infiltration into tumors, and in some patients, tumor necrosis (Ishikawa et al., 2005; Nagato et al., 2012; Nieda et al., 2004). iNKTs were shown to recognize and kill virally-transfected, CD1d+ NB cells when stimulated with aGC, but could not directly induce such killing when cell lines were CD1d-negative in in vitro killing assays (Metelitsa et al., 2001). This is relevant because NB is characteristically CD1d-negative. However, in elegant co-culture studies, aGC-activated iNKTs have been shown to enhance the NK antibody-dependent cellular cytotoxicity (ADCC) of NB cell lines when anti-GD2 antibodies were provided (Mise et al., 2016). In addition to enhancing NK-cell killing, iNKTs can reinvigorate exhausted CD8+ T cells. For instance, in melanoma, aGC-pulsed APCs were administered to humanized mice xenografted with PD-1 inhibitor resistant melanoma. This restored cytotoxic activity of exhausted CD8+ T lymphocytes via IL-2 and IL-12 production, and resulted in reduced tumor progression and improved survival (Bea et al., 2018). iNKTs are therefore capable of enhancing an immune response to CD1d-tumors, such as NB, when activated with GAg or GAg-loaded APCs. However, given that many metastatic solid tumors including NB are associated with lower levels of iNKTs (limiting the potential benefits of mere iNKT activation for these patients), options for iNKT therapies that boost iNKT numbers and activation have been sought.

TABLE 2
iNKT-based therapies in clinical trials

| Therapy | Mechanism of action | Malignancies | References |
|---|---|---|---|
| iNKTs stimulated | Activation of iNKTs | Prostate cancer, HNSCC, melanoma, | 59-62 |
| with free GAg, | via invariant TCR | esophageal, breast cancer, thymus, non- | |
| CD1d-GAg, or | binding | small-cell lung cancer, stomach cancer, | |
| GAg-pulsed | | kidney sarcoma, bladder cancer, | |
| APCs | | pancreatic cancer, prostate cancer, | |
| | | cholangiocarcinoma, colon cancer | |
| Adoptive transfer | Expanded and | HNSCC | 67, 68 |
| of activated | activated iNKTs | | |
| iNKTs | target CD1d+ tumors | | |
| | and/or CD1d+ TAM | | |
| | and MDSC, and | | |
| | recruit NK and T- | | |
| | cells | | |
| GD2-directed | Recognition of | Neuroblastoma | 70 |
| chimeric antigen | neuroblastoma cells | | |
| receptor (CAR)- | and constitutive | | |
| expressing | activation of iNKTs | | |
| iNKTs | with GD2 antigen | | |
| | recognition | | |

Abbreviations: Antigen-presenting cells (APCs); glycolipid antigens (GAg), head and neck squamous cell carcinoma (HNSCC); invariant natural killer T-cell (iNKT).

### E. Adoptive Transfer of INKTs

Adoptive transfer of iNKTs has been attempted in numerous preclinical and clinical studies in NB and other solid tumors. The importance of iNKTs in tumor immunity in NB was demonstrated in iNKT-deficient and iNKT-replete mice xenografted with NB, with the iNKT-replete mice developing significantly fewer metastases and having longer survival than iNKT-deficient mice (Courtney et al., 2017). When iNKTs were adoptively transferred to humanized NOD-Scid-IL2Rgamma^{null} (NSG) mice with NB xenografts, TAMs were reprogrammed from the immunosuppressive, pro-tumor M2 phenotype to the pro-inflammatory, anti-tumor M1 phenotype. Despite this reprogramming, NB tumors progressed, and adoptive transfer of iNKTs resulted in increased PD-L1 expression on M1 and M2 TAMs (Courtney, 2016). Given that iNKTs increase their PD1 expression on activation, there is reason to hypothesize that adjunctive use of PD1/PD-L1 inhibitors could prove useful in improving efficacy of iNKTs responses against NB. In addition to the data on adoptive transfer of iNKTs in NB, iNKT adoptive transfer has been shown to reduce liver metastases of melanoma in a mouse model and has also demonstrated disease responses in patients with HNSCC (Kunii et al., 2009; Shin et al., 2001). Taken together, these preclinical NB studies and clinical studies in other solid tumor patients suggest that the adoptive cell transfer of iNKTs may offer a therapeutic and complementary role in NB by targeting TAMs and enhancing or restoring NK- and T-cell cytotoxicity. However, clinical trials of adoptive transfer of unmodified iNKTs have not yet been performed in patients with NB.

### F. CAR-iNKTs

CAR-modified iNKTs offer another area of great promise in the treatment of NB. GD2-specific CAR-iNKTs reduced the tumor volumes of xenografted CD1d-NB tumors in lymphocyte-deficient mice and prolonged survival (Heczey et al., 2014). Additionally, in contrast to a comparison group in which these mice were treated with GD2-CAR T cells, CAR-iNKTs had significantly greater trafficking to NB tumors and resulted in no graft vs. host disease (GVHD), while the CAR T cells showed liver and lung edema and lymphocytic infiltration consistent with GVHD (Heczey et al., 2014). Although the reason for differences in GVHD between the CAR-iNKTs and CAR T cells is unknown, it is postulated that it may be due to the release of Th2-like cytokines by CD4+ CAR-iNKTs. Importantly, CAR-iNKTs retain both their ability to recognize CD1d/GAg complexes as well their cytotoxic activity against immunosuppressive TAMs (Heczey et al., 2014). In a separate study, a subset of CAR-iNKTs that express CD62L were found to have five-fold longer persistence in host mice than CD62L⁻ AR-INKTs (Tian et al., 2016). Artificial antigen presenting cells (aAPCs) were then created and used to enrich for CD62L⁺ iNKTs that were subsequently modified by CARs specific for GD2 and CD19 antigens. The CAR-iNKTs generated from CD62L⁺ enriched iNKTs were used in mice with NB and lymphoma and demonstrated significantly longer in vivo persistence and therapeutic efficacy when compared with CAR-iNKTs generated without CD62L⁺ cell enrichment (Tian et al., 2016).

However, CAR-iNKTs are now being explored in a Phase I clinical trial (GINAKIT2 trial at Baylor) for patients with relapsed or refractory NB. This study aims to identify the maximum tolerated dose of CAR-iNKTs and involves the use of expanded autologous iNKTs modified with a GD2-CAR containing the IL-15 gene. This trial is currently recruiting and early results from 2 patients treated at the lowest dose level show that one patient's disease was stabilized, while the other had a significant partial response without dose-limiting toxicity (Nowers et al., 2019). The iNKTs used in this clinical study are derived from expanded human peripheral blood mononuclear cells that have not been enriched using the aAPCs discussed above, although there are plans to use this enrichment in future studies (Tian et al., 2016).

The potential advantages of CAR-iNKTs over CAR-Ts in NB include their relatively higher NB tumor penetration and reduced incidence of GVHD in pre-clinical models. Additionally, retention of the TCR function on CAR-iNKTs allows for clearance of immunosuppressive TAMs that contribute to tumor growth and refractoriness to immunotherapies. Given that GD2 CAR T cell efficacy has been limited due to decreased persistence and tumor penetration in NB, CAR-iNKTs offer an exciting potential solution for overcoming this barrier (Richards et al., 2018).

iNKTs have features that can be used in the treatment of NB and other solid tumors. In particular, the abilities of iNKTs to kill TAMs and MDSCs, mature dendritic cells, and robustly release pro-inflammatory cytokines to recruit and activate conventional T cells, make these cells powerful tools in the armamentarium for NB therapy. The ability of these cells to break down the barriers that have previously limited CAR T cell therapies in solid tumors (limited T cell persistence and potency, inability to kill tumors without target tumor-associated antigen (TAA) expression, and an immunosuppressive TME), and promising pre-clinical data suggest great potential for iNKT in NB. Treatments options that can activate iNKTs for robust and persistent tumor cytotoxicity are highly desired, and include strategies that use soluble proteins loaded with glycolipid antigens, modification of glycolipid antigens, adoptive cell transfer, CAR-iNKTs, and immunomodulation with checkpoint blockade.

### II. CD1d-GD2 FUSION PROTEIN

In certain embodiments, the present disclosure provides fusion proteins comprising CD1d and anti-GD2 antibody, specifically an anti-GD2 scFv. Also provided here are nucleic acids which encode the fusion protein and expression constructs comprising the nucleic acids.

In certain embodiments, the CD1d comprises soluble CD1d polypeptides and polypeptide fragments, which associates with B2-microglobulin. The CD1d molecule is a member of the family of major histocompatibility complex (MHC) antigen-like glycoproteins which associate with β₂-microglobulin and are expressed at the surface of cortical thymocytes, B cells, dendritic cells, Langerhans cells in the skin, and gastrointestinal epithelial cells. CD1d is mainly expressed on dendritic cells or epithelial cells of the gastrointestinal tract. The CD1 family members arc involved in the presentation of glycolipids as antigens.

Full-length CD1d consists of a signal sequence, an extracellular domain, a transmembrane domain and a cytoplasmic domain. The full-length CD1d polypeptide is 335 amino acids in length (Genbank Accession number NP_001757).

In some aspects, the CD1d comprises a soluble CD1d polypeptide or polypeptide fragment. Specifically, soluble CD1d polypeptides include fragments, variants, or derivative thereof of a soluble CD1d polypeptide. Soluble CD1d polypeptides generally comprise a portion or all of the extracellular domain of the polypeptides, including the a1, a2, and a3 domains. Soluble CD1d polypeptides generally lack some or all of the transmembrane domain and cytoplasmic domain. As one of skill in the art would appreciate, the entire extracellular domain of CD1d may comprise additional or fewer amino acids on either the C-terminal or N-terminal end of the extracellular domain polypeptide. Soluble CD1d polypeptides described herein may have various alterations such as substitutions, insertions or deletions. Exemplary amino acids that can be substituted in the polypeptide include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Corresponding fragments of soluble CD1d polypeptides at least 70%, 75%, 80%, 85%, 90%, or 95% identical to the polypeptides and reference polypeptides described herein are also contemplated.

In certain embodiments, the present fusion protein further comprises a β₂-microglobulin polypeptide, which associates with a soluble CD1d polypeptide or polypeptide fragment. β₂-microglobulin is present on the surface of all nucleated cells as the small extracellular subunit of the major histocompatibility complex (MHC) class I molecule and actively participates in the immune response. For a detailed discussion of B2-microglobulin, see, e.g., Peterson et al., Adv. Cancer Res. 24:115-163 (1977); Sege et al., Biochemistry 20:4523-4530 (1981); which are incorporated by reference herein in their entirety. Full-length β₂-microglobulin is a secreted protein which comprises a signal sequence and Ig-like domain. The full-length β₂-microglobulin polypeptide is 119 amino acids in length (Genbank accession number NP_004039).

### A. Anti-GD2 Antibody

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a hetero-tetrameric glycoprotein composed of two identical light (L) chains and two identical heavy (H) chains. An IgM antibody consists of 5 basic hetero-tetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange. Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk. J. Mol. Biol. 196:901-917 (1987)); and/or those residues from a “hypervariable loop” /CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation contrasts with a somatic mutation, which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a stepwise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press. Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies, and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry, In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether two antibodies that compete for binding recognize the same epitope.

In another aspect, the antibodies may be defined by their variable sequence, which include additional “framework” regions. These are provided in Tables 1 and 2 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below).

“Homology” refers to the percent identity between two polynucleotide moieties. Two polynucleotide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified polynucleotide sequence.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP. BESTFIT. BLAST. FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error prone. The sequence analysis tool IgBLAST (world-wide-web at ncbi.nlm.nih.gov/igblast/) identifies matches to the germline V. D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T and X determine the sensitivity and speed of the alignment. The BLASTN program (for nucleotide sequences) uses as defaults a word length (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Yet another way of defining an antibody is as a “derivative” of any of the below-described antibodies and their antigen-binding fragments. The term “derivative” refers to an antibody or antigen-binding fragment thereof that immunospecifically binds to an antigen, but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc. In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art (for example, see Shields. R. L. et al. (2002). J. Biol. Chem. 277(30): 26733-26740; Davies J. et al. (2001), Biotechnology & Bioengineering 74(4): 288-294). Methods of altering carbohydrate contents are known to those skilled in the art, see, e.g., Wallick, S. C. et al. (1988), J. Exp. Med. 168(3): 1099-1109; Tao, M. H. et al. (1989), J. Immunol. 143(8): 2595-2601; Routledge. E. G. et al. (1995), Transplantation 60(8):847-53; Elliott, S. et al. (2003), Nature Biotechnol. 21:414-21; Shields, R. L. et al. (2002), J. Biol. Chem. 277(30): 26733-26740).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### B. Expression Vectors

Various genetic constructs are available that contain the present fusion proteins, and these may be introduced into vectors for expression. Nucleic acids according to the present disclosure that encode prodrug molecules may be optionally linked to other protein sequences. Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

As used herein, the term “polynucleotide” refers to a nucleic acid molecule that either is recombinant or has been isolated free of total genomic nucleic acid. By “isolated” when referring to a nucleotide sequence, is meant that the indicated molecule is present in the substantial absence of other biological macromolecules of the same type. Included within the term “polynucleotide” are recombinant vectors, including, for example, plasmids, cosmids, phage, viruses, and the like. Polynucleotides include, in certain aspects, regulatory sequences, isolated substantially away from their naturally occurring genes or protein coding sequences. Polynucleotides may be single-stranded (coding or antisense) or double-stranded, and may be RNA, DNA (e.g., genomic DNA, cDNA, or synthetic DNA), analogs thereof, or a combination thereof. Additional coding or non-coding sequences may, but need not, be present within a polynucleotide.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

### 1. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment.

A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202, 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The identity of tissue-specific promoters or elements, as well as assays to characterize their activity, is well known to those of skill in the art. Examples of such regions include the human LIMK2 gene (Nomoto et al., 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), DIA dopamine receptor gene (Lee, et al., 1997), insulin-like growth factor II (Wu et al., 1997), human platelet endothelial cell adhesion molecule-1 (Almendro et al., 1996). Tumor specific promoters also will find use in the present disclosure.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

### 2. Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### 3. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see Chandler et al., 1997, herein incorporated by reference).

### 4. Termination Signals

The vectors or constructs of the present disclosure will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the disclosure include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### 5. Polyadenylation Signals

In expression, particularly eukaryotic expression, one will typically include a polyadenylation signal to effect proper polyadenylation of the transcript. The nature of the polyadenylation signal is not believed to be crucial to the successful practice of the disclosure, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

### 6. Origins of Replication

In order to propagate a vector in a host cell, it may contain one or more origins of replication sites (often termed “ori”), which is a specific nucleic acid sequence at which replication is initiated. Alternatively, an autonomously replicating sequence (ARS) can be employed if the host cell is yeast.

### 7. Selectable and Screenable Markers

In certain embodiments of the disclosure, cells containing a nucleic acid construct of the present disclosure may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually, the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

### 8. Viral Vectors

The capacity of certain viral vectors to efficiently infect or enter cells, to integrate into a host cell genome and stably express viral genes, have led to the development and application of a number of different viral vector systems (Robbins et al., 1998). Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher's disease, renal disease and arthritis (Robbins and Ghivizzani, 1998; Imai et al., 1998; U.S. Pat. No. 5,670,488). The various viral vectors described below present specific advantages and disadvantages, depending on the particular gene-therapeutic application.

a. Non-Viral Transformation

Suitable methods for nucleic acid delivery for transformation of an organelle, a cell, a tissue or an organism for use with the current disclosure are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

Injection. In certain embodiments, a nucleic acid may be delivered to an organelle, a cell, a tissue or an organism via one or more injections (i.e., a needle injection), such as, for example, either subcutaneously, intradermally, intramuscularly, intravenously or intraperitoneally. Methods of injection of vaccines are well known to those of ordinary skill in the art (e.g., injection of a composition comprising a saline solution). Further embodiments of the present disclosure include the introduction of a nucleic acid by direct microinjection. Direct microinjection has been used to introduce nucleic acid constructs into Xenopus oocytes (Harland and Weintraub, 1985).

Electroporation. In certain embodiments of the present disclosure, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human K-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 92/17598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Calcium Phosphate. In other embodiments of the present disclosure, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1. BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran. In another embodiment, a nucleic acid is delivered into a cell using DEAE-dextran followed by polyethylene glycol. In this manner, reporter plasmids were introduced into mouse myeloma and erythroleukemia cells (Gopal, 1985).

Sonication Loading. Additional embodiments of the present disclosure include the introduction of a nucleic acid by direct sonic loading. LTK⁻ fibroblasts have been transfected with the thymidine kinase gene by sonication loading (Fechheimer et al., 1987).

Liposome-Mediated Transfection. In a further embodiment of the disclosure, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRI) or Superfect (Qiagen).

Liposome-mediated nucleic acid delivery and expression of foreign DNA in vitro has been very successful (Nicolau and Senc, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the disclosure, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor-Mediated Transfection. Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present disclosure.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present disclosure, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, has been incorporated into liposomes and observed an increase in the uptake of the insulin gene by hepatocytes (Nicolau et al., 1987). It is contemplated that the tissue-specific transforming constructs of the present disclosure can be specifically delivered into a target cell in a similar manner.

### 9. Expression Systems

Numerous expression systems exist that comprise at least a part or all of the compositions discussed above. Prokaryote- and/or eukaryote-based systems can be employed for use with the present disclosure to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. pGEM-T Easy vectors, pCon Vectors™, Lonza pConIgG1 or pConK2 plasmid vectors, and 293 Freestyle cells or Lonza CHO cells are also useful for expression of the disclosed prodrug constructs.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but arc protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary (CHO), W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr that confers resistance to; gpt, which confers resistance to mycophenolic acid; neo, that confers resistance to the aminoglycoside G418; and hygro, which confers resistance to hygromycin.

### 10. Purification

In certain embodiments, the interferon prodrugs of the present disclosure may be purified. The term “purified,” as used herein, is intended to refer to a composition, isolatable from other components, wherein the protein is purified to any degree relative to its naturally obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include precipitation with ammonium sulfate, PEG, antibodies and the like or by heat denaturation, followed by centrifugation; gel filtration, reverse phase, hydroxylapatite and affinity chromatography; and combinations of such and other techniques.

In purifying a peptide of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide. Commonly, antibodies are fractionated utilizing agents (i.e., protein A) that bind the Fc portion of the antibody. Where the interferon prodrug contains such a domain, this approach could be used.

Various methods for quantifying the degree of purification of the protein or peptide will be known to those of skill in the art in light of the present disclosure. These include, for example, determining the specific activity of an active fraction, or assessing the amounts of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### C. Conjugates

The present fusion proteins may be linked or covalently bound or complexed to at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which may be attached to the fusion protein include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which may be conjugated to the fusion protein include enzymes, radiolabels, haptens, fluorescent labels, phosphorescent molecules, chemiluminescent molecules, chromophores, photoaffinity molecules, colored particles or ligands, such as biotin.

Fusion proteins may also comprise diagnostic agents, for example, by coupling imaging agents for use in vivo diagnostic protocols. The imaging moieties used can be paramagnetic ions, radioactive isotopes, fluorochromes, NMR-detectable substances and X-ray imaging agents. In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

Radioactively labeled conjugates of the present disclosure may be produced according to well-known methods in the art. For instance, fusion proteins can be iodinated by contact with sodium and/or potassium iodide and a chemical oxidizing agent such as sodium hypochlorite, or an enzymatic oxidizing agent, such as lactoperoxidase. Fusion proteins according to the disclosure may be labeled with technetium 99 by ligand exchange process, for example, by reducing pertechnate with stannous solution, chelating the reduced technetium onto a Sephadex column and applying the peptide to this column. Alternatively, direct labeling techniques may be used, e.g., by incubating pertechnate, a reducing agent such as SNCh, a buffer solution such as sodium-potassium phthalate solution, and the peptide. Intermediary functional groups which are often used to bind radioisotopes which exist as metallic ions to peptide are diethylenetriaminepentaacetic acid (DTPA) or ethylene diaminetetracetic acid (EDTA).

### III. PHARMACEUTICAL COMPOSITIONS AND METHODS OF TREATMENT

### A. Pharmaceutical Compositions

The present disclosure provides pharmaceutical compositions comprising a CD1d-anti-GD2 fusion protein, C34 glycolipid, and/or iNKT cells activated with the fusion protein. Such compositions comprise a prophylactically or therapeutically effective amount of the peptide/polypeptide and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the fusion protein, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Fusion proteins of the present disclosure, as described herein, can be formulated for parenteral administration, e.g., formulated for injection via the intradermal, intravenous, intramuscular, subcutaneous, intra-tumoral or even intraperitoneal routes. The fusion proteins could alternatively be administered by a topical route directly to the mucosa, for example by nasal drops, inhalation, or by nebulizer. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### B. Methods of Treatment

Further provided herein are methods for treating or delaying progression of cancer in an individual comprising administering to the individual an effective amount of a fusion protein or iNKT cells activated by the fusion protein provided herein to a subject with cancer. The subject may have increased expression of GD2, or other cancer targets overexpressed on cancer cell surface, and/or cancer targets present in tumor microenvironment. The cancer may be neuroblastoma, particularly pediatric neuroblastoma.

Examples of cancers contemplated for treatment include colorectal cancer, lung cancer, head and neck cancer, breast cancer, prostate cancer, renal cancer, bladder cancer, testicular cancer, ovarian cancer, cervical cancer, pancreatic cancer, liver cancer, other gastrointestinal cancers, bone cancer, lymphomas, and pre-neoplastic lesions in these organs.

In some embodiments, the subject is a mammal, e.g., a primate, preferably a higher primate, e.g., a human (e.g., a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

“Prognosis” refers to as a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable survival following cancer treatments, such as a conventional cancer therapy.

The term “subject” or “patient” as used herein refers to any individual to which the subject methods are performed. Generally, the patient is human, although as will be appreciated by those in the art, the patient may be an animal. Thus, other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of patient.

“Treatment” and “treating” refer to administration or application of a therapeutic agent to a subject or performance of a procedure or modality on a subject for the purpose of obtaining a therapeutic benefit of a disease or health-related condition. For example, a treatment may include administration chemotherapy, immunotherapy, radiotherapy, performance of surgery, or any combination thereof.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

Likewise, an effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The materials identified by the disclosed methods will be useful in treating cancers. Types of cancers to be treated with the binding agents of the disclosure include, but are not limited to, hematological cancers, solid tumors, and non-solid tumors. Examples of solid tumors, such as sarcomas and carcinomas, include fibrosarcoma, myosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma. CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases). Adult tumors/cancers and pediatric tumors/cancers are also included.

The subject/patient may be an animal or any species of mammal, including, without limitation, a horse, a dog, a cat, a pig, or a primate. In a particular embodiment, the subject/patient is a human.

The ability of the present fusion proteins to modulate an immune response can be readily determined by an in vitro assay. iNKTs for use in the assays include transformed iNKT cell lines, or iNKTs which are isolated from a mammal, e.g., from a human or from a rodent such as a mouse. iNKTs can be isolated from a mammal by sorting cells that bind CD1d:α-GalCer tetramers. See, for example. Benlagha et al., J Exp Med 191 (2000), pp. 1895-1903; Matsuda et al., J Exp Med 192 (2000), pp. 741-754; and Karadimitris et al., Proc Natl Acad Sci USA 98 (2001), pp. 3294-3298. A suitable assay to determine if a fusion protein is capable of modulating the activity of iNKT cells is conducted by coculturing iNKTs and antigen presenting cells, adding the particular compound of interest to the culture medium that targets either the antigen presenting cells or the iNKT cells directly, and measuring IL-4 or IFN-γ production. A significant increase or decrease in IL-4 or IFN-γ production over the same co-culture of cells in the absence of the present fusion protein or, preferably, in the presence of the present fusion proteins with a non-targeting antibody indicates stimulation or inhibition of iNKTs.

The iNKTs employed in the assays may be incubated under conditions suitable for proliferation. For example, an iNKT hybridoma is suitably incubated at about 37° C. and 5% CO2 in complete culture medium (RPMI 1640 supplemented with 10% FBS, penicillin/streptomycin, L-glutamine and 5×10−5 M 2-mercaptoethanol). Serial dilutions of the compound can be added to the iNKT cell culture medium. Suitable concentrations of the compound added to the iNKTs typically will be in the range of from 10⁻¹² to 10⁻⁶M. Use of antigen dose and APC numbers giving slightly submaximal iNKT cell activation is preferred to detect stimulation or inhibition of iNKT responses by the present fusion proteins.

Alternatively, rather than measurement of an expressed protein such as IL-4 or IFN-γ, modulation of NKT activation can be determined by changes in antigen-dependent T cell proliferation as measured by radiolabelling techniques as are recognized in the art. For example, a labeled (e.g., tritiated) nucleotide may be introduced to an assay culture medium. Incorporation of such a tagged nucleotide into DNA serves as a measure of T cell proliferation. This assay is not suitable for iNKTs that do not require antigen presentation for growth, e.g., iNKT cell hybridomas. A difference in the level of T cell proliferation following contact with the compound of the invention indicates the complex modulates activity of the T cells. For example, a decrease in NKT proliferation indicates the fusion protein can suppress an immune response. An increase in NKT proliferation indicates the fusion protein can stimulate an immune response.

Additionally, the 51Cr release assay can be used to determine cytotoxic activity.

These in vitro assays can be employed to select and identify CD1d fusion proteins that are capable of modulating an immune response. Assays described above, e.g., measurement of IL-4 or IFN-γ production or iNKT proliferation, are employed to determine if contact with the compound modulates T cell activation.

In vivo assays also may be suitably employed to determine the ability of a fusion protein of the present disclosure to modulate the activity of NKTs. For example, a compound of interest can be assayed for its ability to stimulate NKT activation or inhibit tumor growth. For example, a fusion protein of the present disclosure can be administered to a mammal such as a mouse, before or after challenge with a tumorigenic dose of transformed cells and the presence or size of growing tumors may be monitored.

### C. Combination Therapies

In certain embodiments, the compositions identified by the presently disclosed methods may be used in combination with at least one additional therapy. The additional therapy may be radiation therapy, surgery (e.g., lumpectomy and a mastectomy), chemotherapy, gene therapy, DNA therapy, viral therapy, RNA therapy, another immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An additional therapy may be administered before, during, after, or in various combinations relative to the T Cell Receptor Therapy described herein. The administrations may be in intervals ranging from concurrently to minutes to days to weeks. In some embodiments where the therapies are provided to a patient separately, one would generally ensure that a significant period of time did not expire between the time of each delivery, such that the two treatments would still be able to exert an advantageously combined effect on the patient. In such instances, it is contemplated that one may provide a patient with both therapies within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below a GD-2 bi-specific iNKT cell engager is “A” and another anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any therapy of the present embodiments to a patient will follow general protocols for the administration of such compounds, taking into account the toxicity, if any, of the agents. Therefore, in some embodiments there is a step of monitoring toxicity that is attributable to combination therapy.

Chemotherapy. A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

Radiotherapy. Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Immunotherapy. The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, crb B, and p155. An alternative aspect of immunotherapy is to combine anticancer effects with immune stimulatory effects. Immune stimulating molecules also exist including cytokines, such as IL-2, IL-4, IL-12, GM-CSF, gamma-IFN, chemokines, such as MIP-1. MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons ÿ, ÿÿ and ÿ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224 Nivolumab, also known as MDX-1106-04, MDX-1106. ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1. MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### IV. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Development of a β-2 Microglobulin-CD1d-anti-GD2 scFv Fusion Protein for Activation and Tumor-Targeting of Invariant Natural Killer T-Cells

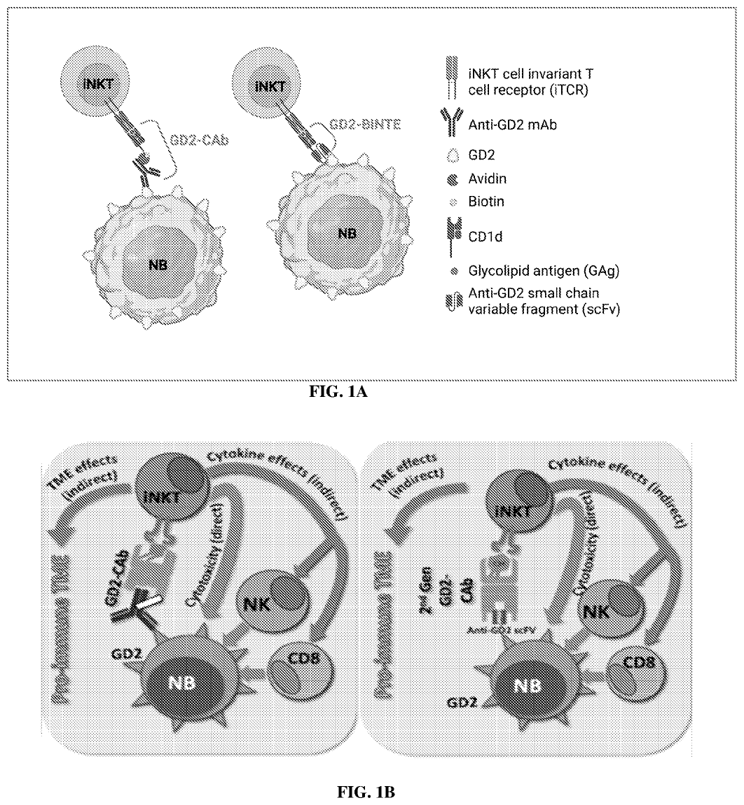

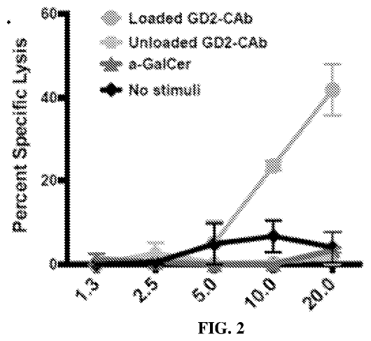

Studies demonstrated the capacity for iNKT activation and NB-specific cytotoxicity with a conjugate protein linking glycolipid antigen-loaded CD1d to an anti-GD2 antibody 14G2a, via streptavidin-biotin linkage. This CD1d-antibody (CAb) tool compound was shown to mediate NB specific lysis (FIG. 2). A pilot in vivo experiment compared treatment of NB tumor-bearing mice with GD2-directed CAb (vs control PBS-treated mice); these preliminary studies showed a trend towards increased survival of CAb-treated mice when compared to control mice. This lack of in vivo efficacy was hypothesized to be due to immune clearance of the CAb molecule, due to the bulky streptavidin/biotin linker required for its generation. However, use of a fusion protein without a streptavidin-biotin linker has potential for significantly less immunogenicity (FIG. 1). Thus, sCD1d-anti-GD2scFv fusion proteins, termed 2^{nd}-Generation CAbs, or Bispecific invariant natural killer T cell engagers (BiNTEs) were designed. (“BiNTE” and “2^{nd} generation CAbs” are used interchangeably within this document).

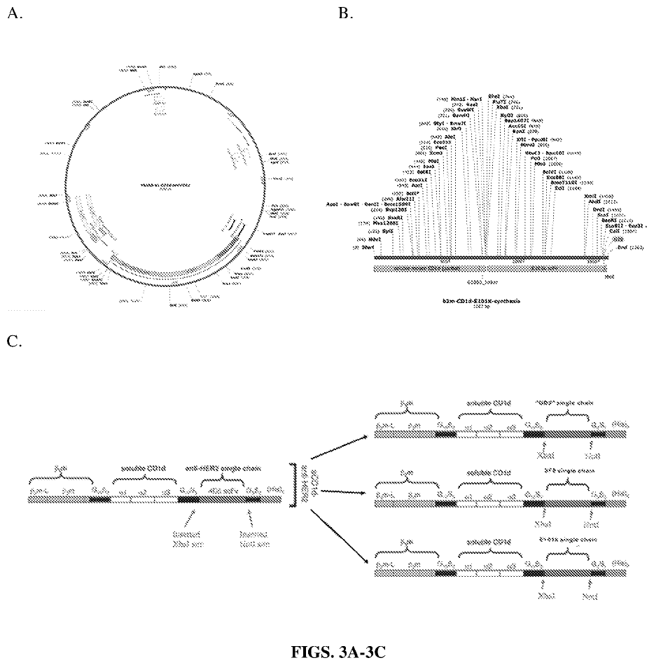

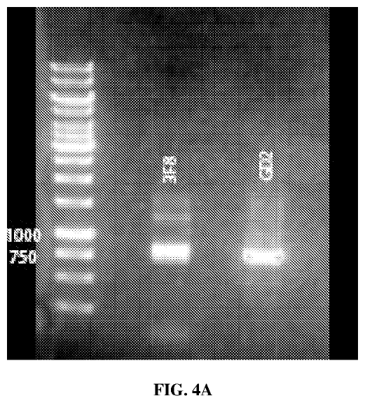

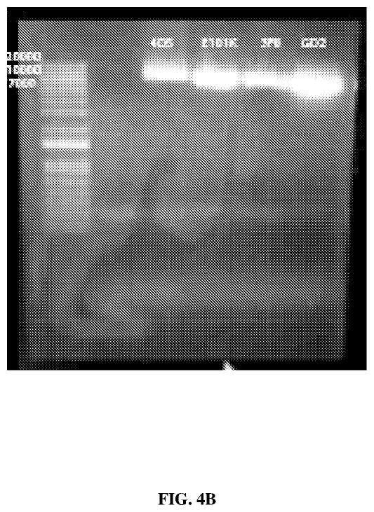

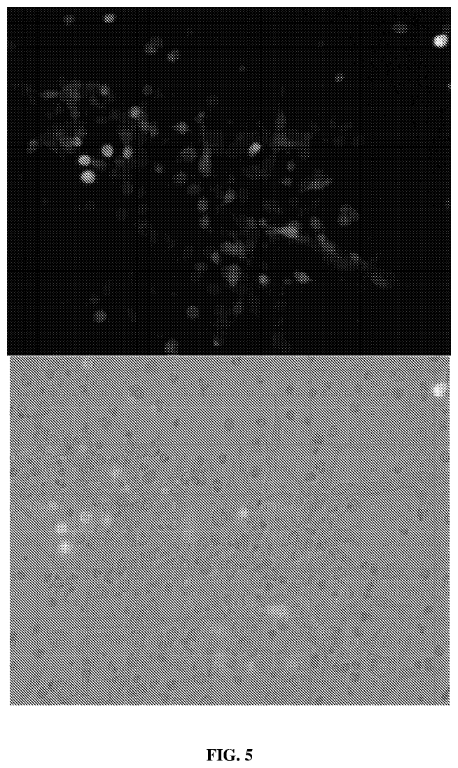

A schematic for cloning is demonstrated in FIG. 3, which shows the pEAK8-4D5 plasmid, and the designed sCD1d-E101K insert with XbaI restriction site for easy scFv manipulation in future cloning. The 3 novel fusion genes are shown in FIG. 3B, containing the 3F8, GD2, and E101K anti-GD2 scFvs. PCR amplification of the 3F8 and GD2 genes were successful and were found at the expected molecular mass on the DNA gel (FIG. 4A). Following ligation, transformation and maxi-kit preparations, pEAK8-4D5, pEAK8-3F8, pEAK8-GD2, and pEAK8-GD2 plasmids were found to be the expected 8100 bp size (FIG. 4B). Sequencing reactions sent to Genewiz confirmed to have desired His-Tag, anti-GD2 scFv. CD1d, and β2m using Snapgene software. Transfection of HEK293T cells had an efficiency of approximately 50%, based upon PEI transfection of p-β-Act-GFP plasmid into HEK293Ts, using cells cultured in the same sequence as the pEAK8 transformed plasmids (FIG. 5). Coomassie staining showed a thick band at 50 kDa in the run-through and wash samples that faded in intensity in the elution samples but remained present. There was also a band at approximately 75 kDa which corresponded to the expected of the sCD1d-anti-GD2scFv protein products and the original sCD1d-anti-HER2 protein products (FIG. 6). Western blots were run to evaluate for the presence of 6×-His. 6×His was identified on proteins created from the pEAK8-4D5 plasmid and the pEAK8-GD2 plasmid, but not the pEAK8-3F8 or pEAK8-E101K protein samples.

TABLE 3
Concentrations of Elutions for Fusion Proteins.

| Sample | Concentration | | Total Protein | |
|---|---|---|---|---|
| sCD1d-4D5 elution 1 | 81.2 | μg/ml | 243.6 | μg |
| sCD1d-4D5 elution 2 | 41.2 | μg/ml | 123.6 | μg |
| sCD1d-E101k elution 1 | 72.85 | μg/ml | 218.55 | μg |
| sCD1d-E101k elution 2 | 54.79 | μg/ml | 164.37 | μg |
| sCD1d-GD2 elution 1 | 77.57 | μg/ml | 232.71 | μg |
| sCD1d-GD2 elution 2 | 55.5 | μg/ml | 166.5 | μg |
| sCD1d-3F8 elution 1 | 65.54 | μg/ml | 196.62 | μg |
| sCD1d-3F8 elution 2 | 74.25 | μg/ml | 222.75 | μg |

Concentrations of elution samples 1 and 2 as determined by Bradford assay. 3 ml of each elution was obtained so total protein is concentration of each elution sample × 3 ml.

The results demonstrated the successful generation of three sequence-confirmed plasmids containing a novel fusion between β2m, sCD1d, and three separate anti-GD2 scFvs of varying affinity (GD2, 3F8, and E101K). These fusion proteins are used in in vitro and in vivo experiments to test their ability to mediate iNKT cytokine release and iNKT-driven NB-cytotoxicity, and to prolong survival in an NB mouse model.

The transfection of HEK293T cells was approximately 50%, which is adequate for sufficient protein production but may be optimized by adjusting the ratio of nitrogen in the PEI to phosphorous in the DNA as was described previously, or via using an alternative transfection method (Huh et al., 2007). The PEI caused some rounding up of the HEK293T cells and is known to be toxic. 293Fectin™ (Thermo Fisher) is used to increase transfection efficiency and obtain a greater and more pure protein yield. All three of the fusion sCD1d-anti-GD2scFv proteins were demonstrated to produce proteins that tracked at the expected molecular mass of 75 kDa on Coomassie gels, as did the CD1d-anti-HER2scFv protein. However, the elution was not completely pure and the main contaminant appeared at 50 kDa, which is most likely albumin from the 2% FBS-containing expression media used for the HEK293T cells. However, only two out of the four fusion proteins, CD1d-GD2 and CD1d-4D5, had detectable 6×His-tags. The reason for the lack of 6×His detection in the sCD1d-3F8 and sCD1d-E101K plasmids is unclear but may be related to incomplete transfer of protein on the Western blot sample or inadequate staining. The 6×His signal was confirmed to be present in the pEAK8-E101Kand pEAK8-3F8 plasmid preparations.

A suspension cell culture of embryonic kidney cells (293F) in serum free expression media (Freestyle media) is used to reduce albumin contamination of the collected supernatant. These fusion proteins are used in in vitro killing assays with iNKT cells as effectors and neuroblasts as targets. Additionally, these proteins are used to determine whether these fusion proteins activate iNKTs in vivo, and whether they can mediate control of NB growth in an immunocompetent mouse model of NB, the transgenic TH-MYCN^{+/+} mouse. These mice are immunocompetent hosts that, by virtue of forced expression of MYCN under a tyrosine hydroxylase promoter, spontaneously develop NB tumors at autochthonous sites and with genetic and ultrastructural identity with that found in human disease.

### Example 2—Materials and Methods

DNA Plasmid templates and Primers. The pEAK8 vector containing genes for β2m, sCD1d, an anti-HER2 scFv called 4D5, a 6×His tag and ampicillin resistance, (pEAK8-4D5), was a gift from Dr. Alena Donda of the University of Lausanne, Switzerland (FIG. 3a). pTRPE plasmid containing m3F8 (pTRPE-m3F8) and pTRPE plasmid containing anti-GD2 scFv derived from the 14G2a antibody called “GD2” (pTRPE-GD2) were received through a collaboration with Dr. Sarah Richman and Dr. Mike Milone at the University of Pennsylvania. A GFP containing plasmid (p-β-Act-GFP) was obtained as a gift from Dr. Ted Hofman at the Children's Hospital of Philadelphia. Forward and reverse primers for the pTRPE-3F8 and pTRPE-GD2 were designed for PCR amplification of the 3F8 and GD2 genes and inclusion of desired restriction sites. Forward primers for pTRPE-3F8 (3F8For) and pTRPE-GD2 (GD2For) contained a restriction site for XbaI restriction enzyme. Reverse primers for pTRPE-3F8 (3F8-Rev) and pTRPE-GD2 (GD2-Rev) contained a restriction site for NotI restriction enzyme. pEAK8-β2m-sCD1d-anti-GD2scFv-6×His plasmids are referred to as pEAK8-E101K, pEAK8-3F8, and pEAK8-GD2.

Cloning of 2-microglobulin-CD1d-scFv fusions genes. Generation of pEAK8 plasmid containing β2m. CD1d, and anti-GD2 scFv E101K: First, a DNA sequence was designed containing the anti-GD2 scFv E101K, linked to a partial sequence of CD1d via a G10S3 linker. A restriction site for XhaI was included at the start of the E101K scFv sequence, so that the scFv region could be easily manipulated (FIG. 3). This sequence was synthesized by Genewiz labs (Genewiz.com). The sCD1d-E101K fragment and the original pEAK8 plasmid were digested with NcoI and NotI enzymes. Subsequently, a ligation reaction was performed to insert the synthesized CD1d-E101K gene fragment; this was confirmed by heat shock transfection of competent TopTen E. coli (Thermofisher) via growth on ampicillin LB-agar plates. Maxikit (Qiagen) preparation was done for use of pEAK8-4D5 and pEAK8-E101K plasmids in further cloning and protein purification. The sequence was confirmed as described in quality assessment section below.

Generation of pEAK8 plasmids containing the anti-GD2 scFvs, 3F8 and GD2: PCR reactions were performed using 3F8For and 3F8 Rev primers on pTRPE-3F8 template, and GD2For and GD2Rev on pTRPE-GD2 template. Size of PCR products was confirmed with gel electrophoresis (FIG. 4), and 3F8 and GD2 inserts were isolated using QIAquick PCR purification kit (Qiagen). 3F8 and GD2 inserts, along with the pEAK8-E101K, and 6×His tag sequence were digested using NotI and XbAI restriction enzymes. The GD2 and 3F8 inserts were then independently inserted into the pEAK8 plasmid, generating pEAK8-β2m-CD1d-GD2-6His (pEAK8-GD2) and pEAK8-β2m-CD1d-3F8-6His (pEAK8-3F8) plasmids.

Quality assessment of pEAK8 plasmids: Sanger Sequencing: Sequencing with primers generated for the pEAK 8 plasmid gene sequence called pEAK8For and pEAK8Rev was used to align 5′ to the β2m sequence and 3′ to the 6×His sequence of the pEAK8 plasmids, respectively. Primers were ordered from, and sequence confirmed with, Genewiz Sanger sequencing. Sequence for pEAK8 For: TGC AAA CAG AAG CTG GTC CC (SEQ ID NO:4). Sequence for pEAK8Rev: ACG TGT CAG CAC CCG GCT GGG (SEQ ID NO:5).

Assessment of purity: All pEAK8 plasmids were tested for purity using optical density (OD) readings using Nanodrop (Thermo Scientific). OD 280/260 ratios at or greater than 1.9 were considered adequate.

Transfection of HEK293T cells using Polyethylenimine: Plasmids including pEAK8-E101K, pEAK8-3F8, pEAK8-GD2, pEAK8-4D5, and p-β-Act-GFP were transfected into embryonic kidney cells (HEK293T cell line) in these experiments. HEK293T adherent cell line was grown to approximately 90% adherence in 2% FBS-containing DMEM media in T75 flasks. p-β-Act-GFP transfection was used to determine transfection efficiency. 50 μg of plasmid DNA per sample was then independently combined with polyethylenimine (PEI) (100 μL of 1 mg/ml stock) and 5 ml of serum free DMEM media to create a DNA complex at room temperature. This was left for 10 minutes at room temperature prior to adding 10 ml fresh DMEM media with 2% FBS. The entire mixtures (15 ml) were then added onto the flasks of HEK293T cells and incubated for 4 hours at 37° C. After 4 hours, 10 ml of additional 2% DMEM media was then added to the cells for a total of 25 ml. The HEK293T cells were then left for 2 days to express and secrete protein into the supernatant. On the day of media (supernatant) collection, cells that were transfected with p-β-Act-GFP were visualized under a fluorescence microscope.

Purification of β2m-CD1d-E101K-6×His, β2m-CD1d-3F8-6×His, β2m-CD1d-GD2-6×His, β2m-CD1d-HER2-6×His fusion proteins. Supernatant from HEK293T cells transfected with pEAK8-E101K, pEAK8-GD2, pEAK8-3F8, and pEAK8-4D5 plasmids was collected and centrifuged to remove residual HEK293T cells. Supernatants were filtered through 0.22-micron filters. An aliquot of each of the supernatants were saved for further analysis. The remainder of the supernatants were then applied to HisPure™ Ni-NTA spin columns (Thermo Fisher) with 3 ml resin and placed on a rocking table in the cold room for 30 minutes before being centrifuged and collected in 50 ml conical tubes. Spin columns were centrifuged per protocol and the run-through was then stored for further analysis on Coomassie gels. Three washes were performed of NiNTA resin with wash buffer containing 15 mM imidazole, and each wash was saved. Finally, elution buffer containing 150 mM imidazole was applied to NiNTA columns, spun, collected, and then repeated twice; all samples were saved for analysis on Coomassie gels. All elutions had protein concentrations determined using Bradford Assay (Table 3).

Coomassie gels. All samples saved from above steps were prepared for gel electrophoresis by denaturing via mixing with 4× NuPAGE LDS sample buffer and heating to 70° C. for 10 minutes. Dual Color protein ladder (Bio-Rad) and denatured protein samples were loaded onto 10% Bis-Tris gel, run until loading dye front reached the bottom of the gel. Gels stained with G-250 overnight. Gels were all then de-stained until protein bands were clearly seen.

Western blotting. Protein denaturing gels were run until dye front reached the end of the gel, as above, using β2m-CD1d-E101K, β2m-CD1d-GD2, β2m-CD1d-3F8, and β2m-CD1d-HER2 run through, wash, and elution samples. The gel was then transferred onto a PVDF membrane in transfer buffer. After running for 90 minutes, the PVDF membrane was checked for transfer by looking for complete transfer of the dual color ladder on to the membrane from the gel. PVDF membrane was incubated for 1 hour in blocking buffer (LiCor) before addition of primary antibody (biotin-conjugated anti-6His) in additional blocking buffer, and overnight incubation at 4° C. on an orbital shaker. This was then washed 3 times, prior to applying the secondary antibody. The membrane was stained for 1 hour at room temperature before being washed 3 times. The gel was then visualized on the Odyssey Imaging system (LiCor).

Data Analysis. All genetic sequencing data files were downloaded as .Seq files and analyzed on Snapgene software and compared to published sequences on Uniprot.

### Example 3—Invariant Natural Killer T-Cell Responses to Stimulation With Phenylated Glycolipid Antigen

In the following study, the effects of the phenylated GAg, C34. were evaluated in comparison with the canonical GAg, aGC, on iNKT cell activation in “acute” and “chronic” stimulation scenarios to evaluate for robustness of iNKT cytokine production as well as iNKT anergy. The influence of these GAgs was subsequently tested on iNKT-mediated killing of CD1d+ lymphoma cells.

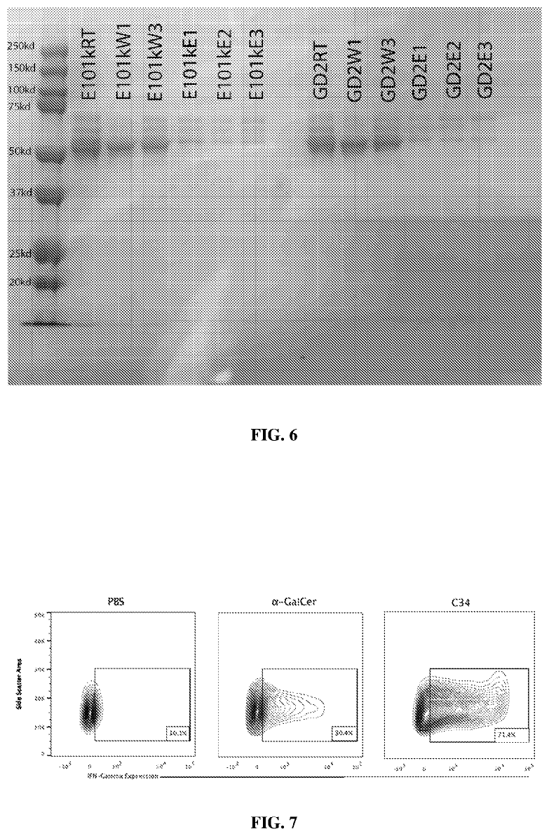

iNKT response to acute GAg stimulation: The expression of IFN-γ in iNKTs isolated from mice injected with C34 and a GC was increased following acute stimulation: C34 (71.4% of iNKTs) and aGC (30.4% of iNKTs), relative to the PBS control (10.1% of iNKTs). Representative IFN-γ expression for acute stimulation is shown in FIG. 7. No significant differences in iNKT expression of surface CD107a or intracellular IL-4 were found when comparing PBS vs aGC or PBS vs C34.

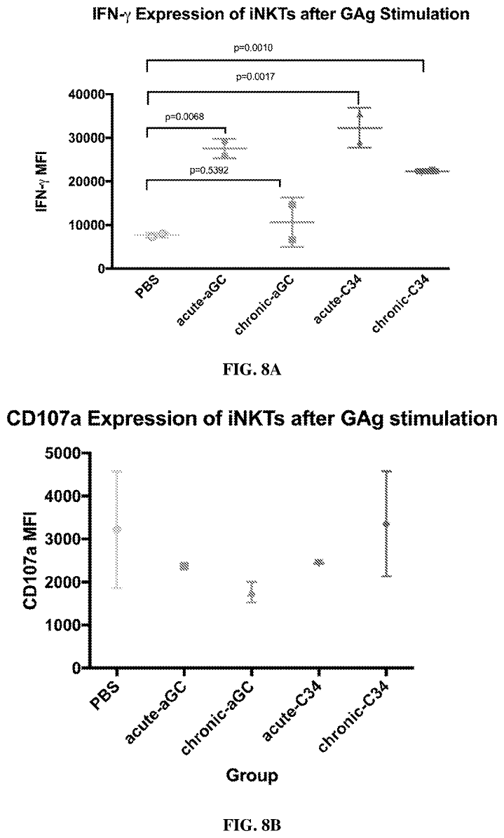

Assessment of anergy in acute versus chronic GAg stimulation: In the acute stimulation protocol, IFN-γ MFI was increased in the aGC and C34 groups relative to the PBS stimulation group (p=0.012 for aGC vs PBS, p=0.0034 for C34 vs PBS, n=2) (FIG. 8A). In the chronic stimulation protocol, in which mice all received three intraperitoneal injections of aGC or C34 over a 7-day period, IFN-γ MFI was greater in the chronic C34 group relative to PBS (p=0.0010, n=2), while there was no difference in the chronic aGC group relative to PBS control (FIG. 8A). There was not a significant difference between the chronic C34 vs acute C34 groups. No significant difference was found between the chronic aGC and chronic C34 groups.

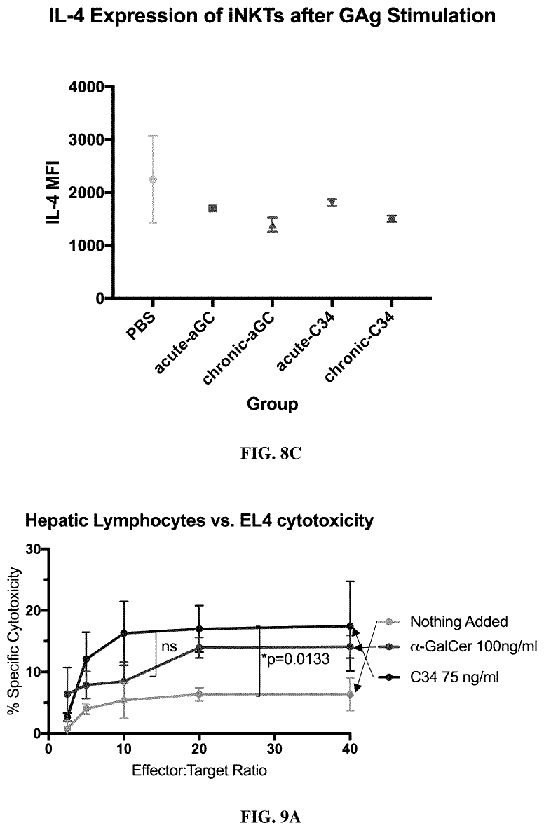

Cytotoxicity of CD1d-expressing lymphoma with hepatic lymphocytes and sorted iNKTs: Unsorted hepatic lymphocytes were demonstrated to mediate higher percent specific lysis in aGC and C34-exposed conditions in comparison with control conditions when hepatic lymphocytes were co-cultured with ⁵¹Cr-treated EL4 lymphoma cell line (p=0.0133 for C34 vs nothing added) (FIG. 9A). The percent specific lysis was not significantly different in the aGC and C34-stimulated hepatic lymphocyte vs EL4 conditions. Sorted murine iNKTs exposed to aGC and C34 also demonstrated greater percent specific lysis relative to control (p=0.014 for C34 vs control) (FIG. 9B). aGC and C34 groups did not have statistically significant differences in percent specific cytotoxicity.

The primary aim of the present study was to evaluate the responsiveness of iNKTs to C34 and aGC glycolipid antigen stimulation. In vivo production of cytokines was measured after acute and chronic stimulation of hepatic iNKTs, and the in vitro assessment of cytotoxicity was assessed via the use of ⁵¹Chromium-release killing assays. The findings indicate that C34 induces robust production of IFN-γ in hepatic iNKTs within hours. However, iNKT IFN-γ production is diminished by chronic aGC treatment to levels similar to that of PBS controls, suggesting that this treatment potently induces anergy. Conversely, chronic stimulation of iNKTs with C34 resulted in IFN-γ levels similar to that of acute aGC treatment, suggesting that C34 stimulation may maintain iNKT responsiveness and avoid anergy. The C34-CD1d-complex has been shown to have a KD value of 2.96+/−0.76 nM for an iNKT hybridoma, while the aGC-CD1d complex had a KD value of 52.51+/−0.09 nM (Wu et al., 2011). As mentioned above, this result is relevant for iNKT-based tumor-directed therapies, as anergy diminishes the anti-tumor potential of iNKTs.

Using killing assay experiments, the implications of C34 and aGC stimulation of iNKT-mediated cytotoxicity were explored. First, using unsorted hepatic lymphocytes, which are enriched for iNKTs, equivalent percent specific lysis were found against EL4 lymphoma cell lines in both aGC and C34 groups, suggesting that C34 and aGC mediate equivalent iNKT-mediated anti-CD1d+ tumor cytotoxicity. This has not been demonstrated previously. Furthermore, there is a trend towards higher percent specific lysis at lower E:T ratios in the C34 group relative to the aGC group in the hepatic lymphocyte killing assays, although this did not reach statistical significance. These results were followed up with killing assays utilizing sorted iNKT cells, demonstrating again specific lysis of EL4s that was equivalent in both groups.

aGC has been used extensively in pre-clinical and clinical trials for stimulation of iNKTs for cytotoxicity, is a known potent induced of iNKT-mediated cytotoxicity of CD1d+ tumor cell lines as well as tumor associated macrophage (TAM) and myeloid derived suppressor cells (MDSC), and has led to tumor necrosis and immune infiltration in tumors when given to patients with various malignancies (Song et al., 2009). Therefore, the equivalent cytotoxicity of C34 to aGC, coupled with the lack of anergy demonstrated on repeated stimulation with C34, demonstrate greater therapeutic efficacy for C34-stimulated iNKT-based therapies relative to aGC-stimulated iNKTs.

iNKT-based anti-tumor therapies can be used to target CD1d+ tumors and immunosuppressive cells in the TME. Further, iNKTs can be targeted to tumor-associated antigens using GAg-loaded CD1d/anti-tumor specific antigen scFv fusion proteins.

### Example 4—Materials and Methods

Primary cells, cell lines, and reagents. Murine hepatic lymphocytes/sorted NKTs: iNKT-enriched cell populations (between 10-30%) were isolated by harvesting livers from C57/BL6 mice in terminal surgeries. Livers were rinsed with RPMI media and then processed through a 70 micron nylon cell strainer (Fisher). The cell suspension was washed twice with 2.5% Fetal Bovine Serum (FBS)-containing RPMI media and spun at 1400rpm in a conical tube for 5 min after each wash. Percoll (GE healthcare) was then used to create a gradient for centrifugation with a gradient of 35% to 75%. The cell pellet was resuspended thoroughly in a the 35% Percoll RPMI solution and then the 75% Percoll RPMI solution was pipetted on the bottom. The lymphocyte coat was then isolated by drawing up the buffy coat into a transfer pipet and transferred to a separate 50 ml conical tube. This sample is spun down and red-cell lysis performed on resuspended cell pellet using ACK lysing buffer (Gibco) on ice for 1 minute. The isolated lymphocytes are then (1) used in killing assays as unsorted hepatic lymphocytes or (2) washed in FACS buffer and stained with anti-NK1.1 and -TCR-β antibodies for flow sorting as described previously (Callea et al., 2015). Stained cells were then sorted on FACSAria Fusion or FACSJazz Sorter for NK1.1, TCR-β double-positive cells.

Peripheral Blood Mononuclear Cells (PBMCs): Deidentified healthy human donor PBMCs were obtained from the Human Immunology Core at the University of Pennsylvania. PBMCs were expanded for 7-8 days using aGC (500 ng/ml), IL-15 (10 ng/ml), and IL-2 (50 U/ml) in 12-well plates with 30 million PBMCs per plate. After 7-8 days of expansion. PBMCs are stained with anti-Vα24 and -CD3 antibodies, following by sorting on FACSAria Fusion Sorter (Becton Dickinson; BD) to obtain a pure human iNKT population.

EL4 lymphoma cell line: was obtained from ATCC and maintained in complete RPMI media (10% FBS, 1% penicillin/streptomycin, 10% L-glutamine) at 37-degree Celsius in 5% CO₂.

Glycolipid antigen-iNKT stimulation experiments. C57/BL6 mice were injected with free glycolipid antigen or phosphate-buffered saline (PBS) intraperitoneally at a dose of 4 μg per injection in “acute” and “chronic” exposure situations. In acute stimulation conditions, intraperitoneal injections were given at hour 0, then at hour 4 mice were sacrificed and livers harvested and processed as above. In chronic stimulation conditions, animals received the 4 μg dose of glycolipid antigen days 0, 4, and 7. Livers were then harvested 4 hours after the final intraperitoneal injection and processed as described above. Hepatic mononuclear cells were extracellularly stained with fluorophore-labeled loaded-CD1d tetramer, anti-NK1.1, anti-CD107a, and then fixed and stained intracellularly with anti-IFN-Y, anti-IL-4, anti-TCR-β using a Fixation/Permeabilization kit (BD) and analyzed on FACSVerse flow cytometer (BD).

⁵¹Cr Killing Assays. EL4 lymphoma cells were used as targets and were labeled with ⁵¹Cr (Perkin-Elmer) at a ratio of 1 μCi per 1×10⁶ cells at 37° C. for 1 hour; excess ⁵¹Cr was removed by extensive washing in RPMI media as described previously (Orange et al., 2022). Murine hepatic iNKT-enriched cell populations and flow cytometrically sorted iNKTs were plated in 96-well round-bottom wells. 2×10⁵ target cells were then mixed with these iNKTs in each well at designated effector to target ratios with 100 ng/mL of glycolipid antigen in triplicate. After 16 hours, supernatants from each well were transferred to a Deepwell Lumiplate (Perkin Elmer), dried overnight, and then analyzed by a TopCount gamma radiation detector (Perkin Elmer). Spontaneous release was determined by collecting supernatants from cultures of targets alone, while maximum lysis was measured from the supernatants of target cells that were fully lysed with detergent. Percent specific lysis was calculated by the equation: 100× (sample lysis cpm-spontaneously released cpm)/(maximum cpm-spontaneously released cpm), as described previously (Orange et al., 2022).

Data Analysis. Cytokinc expression analysis: FCS files were analyzed using Flowjo (TreeStar) software. Mean fluorescence intensity (MFI) of IFN-γ CD107a, and IL-4 on iNKT cells was compared between PBS, aGC, and C34-stimulated groups using Graphpad (Prism) and compared using Student's t-test between two Groups (ex: PBS vs aGC), and one-way ANOVA for comparison of PBS, aGC, and C34.

⁵¹Cr-release killing assays: Percent specific lysis was calculated as described above and triplicate values were analyzed using Graphpad (Prism). Mean values and standard errors of the mean were calculated. Percent specific lysis among the three groups were compared using one-way ANOVA. Significance was set at p value of <0.05.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference.
Bae, et al., Cancer research 78 (2018) 5315-5326.
Bassiri et al., Oncoimmunology 2 (2013) e27440.
Bedard et al., Frontiers in immunology 8 (2017) 1829.
Beldi-Ferchiou & Caillat-Zucman, Int J Mol Sci 18 (2017).
Bellucci et al., Oncoimmunology 4 (2015) e1008824.
Berzins et al., Nat Rev Immunol 11 (2011) 131-42.
Brennan et al., Nature immunology 12 (2011) 1202-11.
Callea et al., Cancer immunology research 3 (2015) 1158-64.
Chang et al., Journal of immunology (Baltimore, Md.: 1950) 181 (2008) 6707-10.
Crowe et al., The Journal of experimental medicine 196 (2002) 119-27.
Coughlan et al., Pediatric hematology and oncology 34 (2017) 320-330.
Courtney et al., Journal of Immunology 198 (2017).
Courtney, The Journal of Immunology 196 (2016).
Cui et al., Science 278 (1997) 1623-6.
Dhodapkar et al., The Journal of experimental medicine 197 (2003) 1667-76.
Durgan et al., Cancer immunology, immunotherapy: CII 60 (2011) 547-58.
Exley et al., Clinical immunology (Orlando, Fla.) 140 (2011) 184-95.
Friedman & Henderson, Children (Basel) 5 (2018).
Fujii et al., Br J Haematol 122 (2003) 617-22.
Fujio et al., J Am Chem Soc 128 (2006) 9022-3.
Giaccone et al., Clinical cancer research: an official journal of the American Association for Cancer Research 8 (2002) 3702-9.
Guillerey et al., Nature immunology 17 (2016) 1025-36.
Guo et al., Oncotarget 7 (2016) 48360-48374.
Heczey et al., Blood 124 (2014) 2824-33.
Hishiki et al., Pediatric surgery international 34 (2018) 169-176.
Hishiki et al., Pediatric surgery international 34 (2018) 195-201.
Hsu et al., The Journal of clinical investigation 128 (2018) 4654-4668.
Huang et al., Journal of immunology (Baltimore, Md.: 1950) 192 (2014) 1972-81.
Huh, et al., Biologicals: journal of the International Association of Biological Standardization 35(2007) 165-71.
Hung et al., Journal of biomedical science 24 (2017) 22.
Ihara et al., Cancer Immunol Immunother 68, 1935-1947 (2019).
Ishikawa et al., Clinical cancer research: an official journal of the American Association for Cancer Research 11 (2005) 1910-7.
Kaneda et al., Biochem Biophys Res Commun 327 (2005) 201-7.
King et al., Frontiers in immunology 9 (2018) 1519.
Kitamura et al., The Journal of experimental medicine 189 (1999) 1121-8.
Kmieciak et al., Journal of immunology (Baltimore, Md.: 1950) 187 (2011) 708-17.
Krasnova et al., Clinical immunology (Orlando, Fla.) 177 (2017) 50-59.
Kunii, et al., Cancer science 100 (2009) 1092-8.
Kuylenstierna et al., European journal of immunology 41 (2011) 1913-23.
Laverdiere et al., Pediatric blood & cancer 45 (2005) 324-32.
Liu et al., The Journal of clinical investigation 122 (2012) 2221-33.
Metelitsa et al., Clinical immunology (Orlando, Fla.) 140 (2011) 119-29.
Metelitsa, et al., Journal of immunology (Baltimore, Md.: 1950) 167 (2001) 3114-22.
Mise, et al., Cancer science 107 (2016) 233-41.
Mody et al., Lancet Oncol 18 (2017) 946-957.
Molling et al., Journal of clinical oncology: official journal of the American Society of Clinical Oncology 25 (2007) 862-8.
Mussai et al., Journal of Immunotherapy 35 (2012) 449-459.
Nagato et al., J Clin Immunol 32 (2012) 1071-81.
Nair & Dhodapkar, Frontiers in immunology 8 (2017) 1178.
Nieda et al., Blood 103 (2004) 383-9.
Nowers et al., Present Positive Early Patient Data From CAR-NKT Neuroblastoma Trial at American Society Of Gene & Cell Therapy 22nd Annual Meeting. (2019).
Orange et al., Proceedings of the National Academy of Sciences of the United States of America 99 (2002) 11351-6.
Parekh, Journal of Clinical Investigation 115 (2005) 2572-2583.
Parckh et al., Journal of immunology (Baltimore, Md.: 1950) 182 (2009) 2816-26.
Portwine et al., Pediatric blood & cancer 63 (2016) 1615-21.
Reilly et al., Cytokine 51 (2010) 227-31.
Richards et al., Frontiers in immunology 9 (2018) 2380.
Shin et al., Int J Cancer 91 (2001) 523-8.
Sohara et al., Cancer research 65 (2005) 1129-35.
Song et al., The Journal of clinical investigation 119 (2009) 1524-36.
Swann et al., Blood 113 (2009) 6382-5.
Tahir et al., Journal of immunology (Baltimore, Md.: 1950) 167 (2001) 4046-50.
Tian et al., The Journal of clinical investigation 126 (2016) 2341-55.
Torreno-Pina et al., Proceedings of the National Academy of Sciences of the United States of America 113 (2016) E772-81.
Tyznik et al., Journal of immunology (Baltimore, Md.: 1950) 181 (2008) 4452-6.
Yu et al., N Engl J Med 363 (2010) 1324-34.
Vinay et al., Journal of immunology (Baltimore, Md.: 1950) 173 (2004) 4218-29.
Waldowska et al., (2), 181-195 (2017).
Wang, et al., Journal of immunology (Baltimore, Md.: 1950) 182 (2009) 6644-7.
Wang et al., J Viral Hepat 20 Suppl 1 (2013) 27-39.
Wesley et al., PLoS Pathog 4 (2008) e1000106.
Wolf, et al., Frontiers in immunology 9 (2018) 384.
Wu et al., 108 (42), 17275-17280 (2011).

1. A composition comprising a CD1d polypeptide conjugated to an anti-GD2 single chain variable fragment (scFv).

2. The composition of claim 1, further comprising a β2-microglobulin (β2m) polypeptide or fragment thereof.

3. The composition of claim 1, wherein the CD1d polypeptide is a soluble CD1d polypeptide or fragment thereof.

4. The composition of claim 1, wherein the anti-GD2scFV has at least 90% sequence identity to the 14G2a GD2 scFv, E101K GD2 scFv, or 3F8 GD2 scFv.

5. The composition of claim 1, wherein the anti-GD2scFV comprises the 14G2a GD2 scFv, E101K GD2 scFv, or 3F8 GD2 scFv.

6. The composition of claim 1, further comprising a peptide linker between the CD1d polypeptide and anti-GD2 scFv.

7. The composition of claim 2, further comprising a peptide linker between the β2m polypeptide and the CD1d polypeptide.

8. The composition of claim 6, wherein the peptide linker is a Glycine-Serine (GS) linker.

9. The composition of claim 6, wherein the peptide linker is G10S3.

10. The composition of claim 1, wherein the composition does not comprise a streptavidin-biotin linker.

11. The composition of claim 1, wherein the CD1d polypeptide is not biotinylated.

12. The composition of claim 1, further comprising a glycolipid antigen.

13. The composition of claim 12, wherein the glycolipid antigen is a non-phenylated glycolipid antigen.

14. The composition of claim 13, wherein the non-phenylated glycolipid antigen is α-GalactosylCeramide (aGC).

15. The composition of claim 12, wherein the glycolipid antigen is a phenylated glycolipid antigen.

16. The composition of claim 15, wherein the phenylated glycolipid antigen is phenyl α-GalactosylCeramide (C34).

17. A polynucleotide encoding the composition of claim 1.

18. The polynucleotide of claim 17, wherein the polynucleotide encodes from N-terminal to C-terminal the β2m polypeptide, the CD1d polypeptide, and the anti-GD2 scFv.

19. The polynucleotide of claim 17, wherein the polynucleotide has at least 90% sequence identity to SEQ ID NO:1, 2, or 3.

20. The polynucleotide of claim 17, wherein the polynucleotide comprises SEQ ID NO: 1, 2, or 3.

21. An expression vector comprising the polynucleotide of claim 17.

22. A host cell comprising an expression vector of claim 21.

23. The host cell of claim 22, wherein the host cell is an antigen presenting cell (APC).

24. The host cell of claim 22, wherein the APC is a dendritic cell.

25. A method of stimulating immune cells comprising said immune cells with the composition of claim 1.

26. The method of claim 25, wherein the immune cells are invariant natural killer T cells (iNKTs).

27. The method of claim 26, wherein the iNKTs are stimulated for less than 24 hours.

28. The method of claim 26, wherein the iNKTs are stimulated for 1-10 days.

29. The method of claim 26, wherein the iNKTs are stimulated more than once.

30. The method of claim 26, wherein the iNKTs are stimulated two, three, or four times.

31. The method of claim 1, wherein the iNKTs are further stimulated with one or more cytokines.

32. The method of claim 31, wherein the one or more cytokines are IL-12 and/or IL-18.

33. A population of GD2-targeted iNKTs stimulated by the composition of claim 1.

34. A pharmaceutical composition comprising the composition of claim 1 and a pharmaceutically acceptable carrier.

35. (canceled)

36. A method for treating cancer in a subject comprising administering an effective amount of the composition of claim 1 to the subject.

37. The method of claim 36, wherein the cancer is neuroblastoma, osteosarcoma, Ewing's sarcoma, and melanoma.

38. The method of claim 36, further comprising administering to said subject at least a second anti-cancer therapy.

39. The method of claim 38, wherein the second anti-cancer therapy is chemotherapy, radiotherapy, gene therapy, surgery, hormonal therapy, anti-angiogenic therapy or immunotherapy.

40. The method of claim 38, wherein the second anti-cancer therapy is a monoclonal antibody, CAR T-cell therapy, or a second bispecific T-cell engager.

41. The method of claim 36, wherein the subject is human.
